SEQUENCE LISTING (23 sequences)

SEQ ID NO: 1 - protein, 11 aa, Artificial Sequence
  source: /note=“Description of Artificial Sequence Synthetic peptide”
HASDSISNSLH

SEQ ID NO: 2 - protein, 7 aa, Artificial Sequence
  source: /note=“Description of Artificial Sequence Synthetic peptide”
  VARIANT (2)..(2): /replace=“Ala”
  VARIANT (6)..(6): /replace=“Glu”
  SITE (1)..(7): /note=“Variant residues given in the sequence have no preference with respect to those in the annotations for variant positions”
YGRQSIQ

SEQ ID NO: 3 - protein, 9 aa, Artificial Sequence
  source: /note=“Description of Artificial Sequence Synthetic peptide”
  VARIANT (4)..(4): /replace=“Ala”
  SITE (1)..(9): /note=“Variant residues given in the sequence have no preference with respect to those in the annotations for variant positions”
QQSESWPLH

SEQ ID NO: 4 - protein, 10 aa, Artificial Sequence
  source: /note=“Description of Artificial Sequence Synthetic peptide”
GFTFSSYTMS

SEQ ID NO: 5 - protein, 16 aa, Artificial Sequence
  source: /note=“Description of Artificial Sequence Synthetic peptide”
TISGGGFGTYYPDSVK

SEQ ID NO: 6 - protein, 10 aa, Artificial Sequence
  source: /note=“Description of Artificial Sequence Synthetic peptide”
  VARIANT (4)..(4): /replace=“Asn”
  SITE (1)..(10): /note=“Variant residues given in the sequence have no preference with respect to those in the annotations for variant positions”
PGYHNYYFDI

SEQ ID NO: 7 - protein, 107 aa, Artificial Sequence
  source: /note=“Description of Artificial Sequence Synthetic polypeptide”
  VARIANT (51)..(51): /replace=“Ala”
  VARIANT (55)..(55): /replace=“Glu”
  VARIANT (92)..(92): /replace=“Ala”
  SITE (1)..(107): /note=“Variant residues given in the sequence have no preference with respect to those in the annotations for variant positions”
EIVLTQSPATLSLSPGERATLSCHASDSISNSLHWYQQKPGQAPRLLIYYGRQSIQGIPARFSGSGSGTDFTLTISSLEPEDFAVYYCQQSESWPLHFGGGTKVEIK

SEQ ID NO: 8 - protein, 119 aa, Artificial Sequence
  source: /note=“Description of Artificial Sequence Synthetic polypeptide”
  VARIANT (102)..(102): /replace=“Asn”
  SITE (1)..(119): /note=“Variant residues given in the sequence have no preference with respect to those in the annotations for variant positions”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYHNYYFDIWGQGTTVTVSS

SEQ ID NO: 9 - protein, 107 aa, Artificial Sequence; identical to SEQ ID NO: 7
  source: /note=“Description of Artificial Sequence Synthetic polypeptide”

SEQ ID NO: 10 - protein, 119 aa, Artificial Sequence; identical to SEQ ID NO: 8
  source: /note=“Description of Artificial Sequence Synthetic polypeptide”

SEQ ID NO: 11 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSIQGIPARFSGSGSGTDFTLTISSLEPEDFAVYYCQQSESWPLHFGGGTKVEIK

SEQ ID NO: 12 - protein, 119 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYNNYYFDIWGQGTTVTVSS

SEQ ID NO: 13 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSASWPLHFGGGTKVEIK

SEQ ID NO: 14 - protein, 107 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSEQGIPARFSGSGSGTDFTLTISSLEPEDFAVYYCQQSASWPLHFGGGTKVEIK

SEQ ID NO: 15 - protein, 445 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYHNYYFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSESWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 445 aa, Artificial Sequence
  source: /note=“Description of Artificial Sequence Synthetic polypeptide”
EVQLVESGGGLVQPGGSLRLSCAASGFTFSSYTMSWVRQAPGKGLEWVATISGGGFGTYYPDSVKGRFTISRDNAKNSLYLQMNSLRAEDTAVYYCARPGYNNYYFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSIQGIPARFSGSGSGTDFTLTISSLEPEDFAVYYCQQSESWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 445 aa, Artificial Sequence; identical to SEQ ID NO: 15
  source: /note=“Description of Artificial Sequence Synthetic polypeptide”

SEQ ID NO: 20 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYGRQSIQGIPARFSGSGSGTDFTLTISSLEPEDFAVYYCQQSASWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, Artificial Sequence
  source: /note=“Description of Artificial Sequence Synthetic polypeptide”
EIVLTQSPATLSLSPGERATLSCHASDSISNSLHWYQQKPGQAPRLLIYYARQSEQGIPARFSGSGSGTDFTLTISSLEPEDFAVYYCQQSASWPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 235 aa, Artificial Sequence
  source: /note=“Description of Artificial Sequence Synthetic polypeptide”
TLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 23 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2024/0141023 A1
(43) Pub. Date: May 2, 2024

(54) METHODS OF TREATING CANCER USING DKK-1 INHIBITORS

(71) Applicant: Leap Therapeutics, Inc., Cambridge, MA (US)

(72) Inventors: Michael H. Kagey, Arlington, MA (US); Girish Somala Naik, New York, NY (US); Cynthia A. Sirad, Walpole, MA (US)

(21) Appl. No.: 17/779,002

(22) PCT Filed: Nov. 23, 2020
(86) PCT No.: PCT/US2020/061773

§ 371 Date: May 23, 2022

Related U.S. Application Data
(60) Provisional application No. 62/939,174, filed on Nov. 22, 2019.

(51) Int. Cl. C07K 16/18; A61K 31/337; A61K 33/243; A61K 39/395; A61P 35/00; C12Q 1/48

(52) U.S. Cl. CPC C07K 16/18; A61K 31/337; A61K 33/243; A61K 39/3955; A61P 35/00; C12Q 1/485; A61K 2039/505; C07K 2317/24

(57) ABSTRACT

A method of treating a subject suffering from a cancer, comprising the steps of obtaining a sample of a cancer cell from the subject; determining a sequence of a phosphatidylinositol 3-kinase catalytic subunit (PIK3CA) protein in the sample; and administering a first amount of a DKK1 inhibitor to the subject determined to have the sequence of PIK3CA protein that includes an activating mutation. The cancer is an epithelial endometrial cancer or an epithelial ovarian cancer.

### RELATED APPLICATION

This application claims the benefit of U.S. Provisional Application No. 62/939,174, filed on Nov. 22, 2019. The entire teachings of the above application(s) are incorporated herein by reference.

### BACKGROUND OF THE INVENTION

Cancer remains an important public health threat with poor prognosis and limited treatment available for many types. There is a significant unmet need for therapies that can increase efficacy in treating cancers, particularly gynecological cancers. The present application provides such therapies.

### SUMMARY OF THE INVENTION

In a first example embodiment, the present invention is a method of treating a subject suffering from a cancer. The method comprises the steps of: obtaining a sample of a cancer cell from the subject; determining a sequence of a phosphatidylinositol 3-kinase catalytic subunit (PIK3CA) protein in the sample; and administering a first amount of a DKK1 inhibitor to the subject determined to have the sequence of PIK3CA protein (SEQ ID NO:23) that includes an activating mutation. The cancer can be an epithelial endometrial cancer or an epithelial ovarian cancer.

In a second example embodiment, the present invention is a method of treating a cancer in a subject in need thereof. The method comprises administering a first amount of a DKK1 inhibitor to the subject, wherein the subject is determined to have an activating mutation of a phosphatidylinositol 3-kinase catalytic subunit (PIK3CA) protein (SEQ ID NO:23). The cancer can be an epithelial endometrial cancer or an epithelial ovarian cancer.

In a third example embodiment, the present invention is a method of treating a subject suffering from a cancer. The method comprises the steps of: obtaining a sample of a cancer cell from the subject; determining a sequence of a phosphatidylinositol 3-kinase catalytic subunit (PIK3CA) protein in the sample; and administering a first amount of a DKK1 inhibitor to the subject determined to have the sequence of PIK3CA protein (SEQ ID NO:23) that includes an activating mutation. The cancer can be an MMMT.

In a fourth example embodiment, the present invention is a method of treating a cancer in a subject in need thereof. The method comprises administering a first amount of a DKK1 inhibitor to the subject, wherein the subject is determined to have an activating mutation of a phosphatidylinositol 3-kinase catalytic subunit (PIK3CA) protein (SEQ ID NO:23). The cancer can be an MMMT.

Another embodiment of the present invention is the use of a DKK1 inhibitor as described herein or a pharmaceutically acceptable salt thereof for the manufacture of a medicament for treating epithelial endometrial cancer or epithelial ovarian cancer in a subject determined to have an activating mutation of a phosphatidylinositol 3-kinase catalytic subunit (PIK3CA) protein (SEQ ID NO:23).

Another embodiment of the present invention is the use of use of a DKK1 inhibitor as described herein or a pharmaceutically acceptable salt thereof for therapy such as for treating epithelial endometrial cancer or epithelial ovarian cancer in a subject determined to have an activating mutation of a phosphatidylinositol 3-kinase catalytic subunit (PIK3CA) protein (SEQ ID NO:23).

Another embodiment of the present invention is the use of a DKK1 inhibitor as described herein or a pharmaceutically acceptable salt thereof for the manufacture of a medicament for treating MMMT in a subject determined to have an activating mutation of of a phosphatidylinositol 3-kinase catalytic subunit (PIK3CA) protein (SEQ ID NO:23).

Another embodiment of the present invention is the use of use of a DKK1 inhibitor as described herein or a pharmaceutically acceptable salt thereof for therapy, such as for treating MMMT in a subject determined to have an activating mutation of a phosphatidylinositol 3-kinase catalytic subunit (PIK3CA) protein (SEQ ID NO:23).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a table representing the amino acid sequence of human PIK3CA (SEQ ID NO:23).

FIG. 2 is a plot (Kaplan-Meier (KM) estimates of Progression Free Survival (PFS) probability vs. time, days post-treatment) that demonstrates a trend for longer median PFS for the patients having a PIK3CA activating mutation.

FIG. 3 is a plot showing the hazard ratio (HR, the risk of having an event that is either “radiographic progression” or “dying” from any cause) computed for the pool of 88 EEC/EOC patients based on PFS outcome in patients that have an activating PIK3CA mutation compared to those who do not have an activating PIK3CA mutation.

FIG. 4 is a plot showing hazard ratios of the same patient pool as in FIG. 3, computed for the pool of 88 EEC/EOC patients based on PFS outcome in patients that have an activating PIK3CA mutation compared to those who do not have an activating PIK3CA mutation, but adjusted for the presence of a Wnt-pathway activating mutation, treatment modality, and tumor type.

FIG. 5 is depicts a plot (KM estimates of Overall (OS) probability vs. time, days post-treatment) that demonstrates a trend for longer median OS for the patients having a PIK3CA activating mutation (median: not reached) compared to those who do not have an activating PIK3CA mutation (median: 365 days).

FIG. 6 is a plot showing the hazard ratio computed for the pool of 88 EEC/EOC patients based on the OS outcome in patients that have an activating PIK3CA mutation compared to those who do not have an activating PIK3CA mutation.

FIG. 7 is a plot showing hazard ratios of the same patient pool as in FIG. 6, computed for the pool of 88 EEC/EOC patients based on OS outcome in patients that have an activating PIK3CA mutation compared to those who do not have an activating PIK3CA mutation, but adjusted for the presence of a Wnt-pathway activating mutation, treatment modality.

FIG. 8 depicts a plot (KM estimates of Progression Free Survival (PFS) probability vs. time, days post-treatment) that shows PFS probability for the patients having none, either one, or both a PIK3CA activating mutation and a Wnt-pathway activating mutation.

FIG. 9 is depicts a plot (KM estimates of Overall Survival (OS) probability vs. time, days post-treatment), and a corresponding table, that shows OS for the patients having none, either one, or both a PIK3CA activating mutation and a Wnt-pathway activating mutation.

FIG. 10 is a plot showing hazard ratios of the same patient pool as in FIG. 9, computed for the pool of 88 EEC/EOC patients based on PFS outcome in patients that have an activating PIK3CA mutation compared to those who do not have an activating PIK3CA mutation, but adjusted for the presence of a Wnt-pathway activating mutation.

FIG. 11 depicts a plot (Progression Free Survival (PFS) probability vs. time, days post-treatment) that shows PFS probability for the patients having a PIK3CA activating mutation and undergoing either a monotherapy or a combination therapy compared to those who do not have an activating PIK3CA mutation.

FIG. 12 depicts a plot (KM estimates of Overall Survival (OS) vs. time, days post-treatment), and a corresponding table, that shows OS probability for the patients having a PIK3CA activating mutation and undergoing either a monotherapy or a combination therapy compared to those who do not have an activating PIK3CA mutation.

### DETAILED DESCRIPTION OF THE INVENTION

A description of example embodiments of the invention follows.

The teachings of all patents, published applications and references cited herein are incorporated by reference in their entirety.

Dickkopf-1 (Dkk-1) is a protein that acts as a natural inhibitor of the canonical Wnt/β-catenin signaling pathway. The Wnt/β-catenin pathway influences a number of biological processes such as cell growth, cell proliferation, stem cell maintenance, cell differentiation, cell polarity, bone development, and adult tissue homeostasis.

In a canonical Wnt/β-catenin signaling pathway, extracellular Wnt ligand binds to its cognate receptor “Frizzled,” and further recruits transmembrane lipoproteins LPR5 and LPR6 (low-density lipoprotein receptor-related proteins 5 and 6) co-receptors. Formation of a Wnt/Frizzled/LPR5/6 complex triggers several intracellular signaling cascades, including the one mediated by the β-catenin protein, a gene product of the CTNNB1 gene. In particular, the formation of a Wnt/Frizzled/LPR5/6 complex results in stabilization of cytoplasmic level of beta-catenin due to the inhibition of the beta-catenin phosphorylation. While phosphorylated beta-catenin is degraded in the cytoplasm, unphosphorylated beta-catenin translocates to the nucleus, where it enhances target gene expression of, e.g., cyclin D1, c-myc, c-jun, cyclooxygenase-2, matrix metalloproteinase-7, vascular endothelial growth factor, and survivin, among other growth factors. Absent the signal from the Wnt/Frizzled/LPR5/6 complex, beta-catenin is phosphorylated by intracellular kinases, such as glycogen synthase kinase 3β (GSK3β) and casein kinas I (CKI). Transduction of a signal from the Wnt/Frizzled/LPR5/6 complex inhibits this phosphorylation.

Extracellular Dkk-1 binds to the LPR5/6 co-receptors and prevents Wnt ligand binding. This results in resuming of beta-catenin phosphorylation and its subsequent degradation, thus inhibiting canonical Wnt signaling pathway.

Phosphoinositide 3-kinases, also called phosphatidylinositol 3-kinases, PI3Ks or PIK3s, are a family of enzymes involved in cellular functions such as cell growth, proliferation, differentiation, motility, survival and intracellular trafficking, which in turn are involved in cancer.

PI3Ks are a family of related intracellular signal transducer enzymes capable of phosphorylating the 3 position hydroxyl group of the inositol ring of phosphatidylinositol (PtdIns), the latter acting as a signal molecule.

PI3Ks have been linked to a diverse group of cellular functions, including cell growth, proliferation, differentiation, motility, survival and intracellular trafficking. Many of these functions relate to the ability of PI3Ks to activate protein kinase B (PKB, also known as AKT) as in the PI3K/AKT/mTOR pathway.

The pleckstrin homology domain of AKT binds directly to PtdIns phosphates, which are produced by activated PI3Ks. Since PtdIns phosphates are restricted to the plasma membrane, this results in translocation of AKT to the plasma membrane. Likewise, the phosphoinositide-dependent kinase-1 (PDK1) also contains a pleckstrin homology domain that binds directly to PtdIns phosphate, causing it to also translocate to the plasma membrane upon PI3K activation. The interaction of activated PDK1 and AKT allows AKT to become phosphorylated by PDK1, leading to partial activation of AKT. Full activation of AKT occurs upon phosphorylation by the TORC2 complex of the mTOR protein kinase.

The PI3K p110α, known as PIK3CA, is mutated in many cancers. Many of these mutations cause the kinase to be more active. PI3K activity contributes significantly to cellular transformation and the development of cancer.

Both PI3K/Akt and Wnt/β-Catenin signaling pathways act as key regulators in cell proliferation, differentiation and growth. Both signaling pathways include GSK3β as a common protein, which mediates an interaction and cross-talk between the pathways.

See URL https://www.uniprot.org/uniprot/P42336 for the amino acid consensus sequence of a human PIK3CA. This sequence is reproduced herein as SEQ ID NO:23.

As used herein, “an activating mutation of PIK3CA protein” refers to a mutation of the genetic sequence encoding PIK3CA protein that changes the amino acid sequence of PIK3CA in a manner that results in a gain of function (e.g., an elevated cellular level of the protein functionally capable of transducing a signal, when compared to a wild type protein, or the protein that is functionally active in the absence of an upstream activating signal, or the protein that is incapable of being functionally attenuated).

Activating mutations may also refer to a noncoding mutation to the PIK3CA genetic locus (e.g., introns, insulators, promoter and enhancers) that result in increased mRNA expression of PIK3CA or increased mRNA stability that results in elevated cellular protein levels of PIK3CA.

The presence of any mutation in a protein can be determined by either one of the following: (1) sequencing the isolated protein of interest and comparing its sequence to the wild type consensus sequence; (2) sequencing the region of the genetic DNA encoding the protein of interest (here, the PIK3CA gene) and translating the nucleotide sequence into a putative amino acid sequence; or (3) sequencing an isolated mRNA or total cellular RNA (e.g. RNA-Seq) containing the transcript of the gene encoding the protein of interest and translating the nucleotide sequence into a putative amino acid sequence. Methods of sequencing peptides and nucleic acids are well known in the art.

Description of mutations of PIK3CA can be found at the URL https://ckb.jax.org/gene/show?geneId=5290. Of particular interest are the activating mutations in the PIK3CA. Examples of such mutations are provided in Table 1:

TABLE 1

| Variant | Impact |
|---|---|
| G12D | missense |
| I20M | missense |
| I31M | missense |
| R38C | missense |
| R38G | missense |
| R38H | missense |
| R38S | missense |
| E39K | missense |
| H47R | missense |
| Y56H | missense |
| Q75E | missense |
| Q75K | missense |
| E78K | missense |
| E80K | missense |
| E81K | missense |
| R88L | missense |
| R88Q | missense |
| R93L | missense |
| R93P | missense |
| R93W | missense |
| E103_G106delinsD | indel |
| E103_P104del | deletion |
| P104L | missense |
| V105_R108del | deletion |
| G106R | missense |
| G106_R108del | deletion |
| G106V | missense |
| R108C | missense |
| R108del | Deletion |
| R108H | missense |
| R108P | missense |
| E109del | Deletion |
| E109_I112delinsD | Indel |
| E110del | deletion |
| E110K | missense |
| K111del | Deletion |
| K111E | missense |
| K111N | missense |
| K111R | missense |
| I112N | missense |
| L113del | Deletion |
| R115L | missense |
| E116K | missense |
| G118D | missense |
| P124L | missense |
| V146I | missense |
| E149G | missense |
| Y165H | missense |
| N170S | missense |
| K179T | missense |
| P217S | missense |
| I273V | missense |
| P298S | missense |
| D300Y | missense |
| T324I | missense |
| L339I | missense |
| V344A | missense |
| V344G | missense |
| V344M | missense |
| N345D | missense |
| N345H | missense |
| N345I | missense |
| N345K | missense |
| N345S | missense |
| N345T | missense |
| N345Y | missense |
| N347K | missense |
| D350G | missense |
| D350N | missense |
| R357Q | Missense |
| G363A | missense |
| G364R | missense |
| E365K | missense |
| E365V | missense |
| P366R | missense |
| P377R | missense |
| C378R | missense |
| C378Y | missense |
| I391M | missense |
| R401Q | missense |
| S405F | missense |
| S405Y | missense |
| I406V | missense |
| C407F | missense |
| C407W | missense |
| C407Y | missense |
| E418K | missense |
| C420G | missense |
| C420R | missense |
| P421L | missense |
| R425L | missense |
| P447_L455del | deletion |
| P449_N457del | deletion |
| P449S | missense |
| P449T | missense |
| H450_P458del | Deletion |
| G451R | missense |
| G451V | missense |
| E453A | missense |
| E453del | deletion |
| E453G | missense |
| E453K | missense |
| E453Q | missense |
| D454Y | missense |
| L456Afs*13 | Frameshift |
| L456R | missense |
| N457K | missense |
| P466S | missense |
| E469delinsDK | Indel |
| P471L | missense |
| P487Q | missense |
| D520V | Missense |
| E522A | missense |
| R524M | missense |
| P539R | missense |
| P539S | missense |
| E542A | missense |
| E542G | missense |
| E542K | missense |
| E542Q | missense |
| E542V | missense |
| E542X | missense |
| T544I | missense |
| T544N | missense |
| E545A | missense |
| E545D | missense |
| E545G | missense |
| E545K | missense |
| E545Q | missense |
| E545V | missense |
| E545X | missense |
| Q546E | missense |
| Q546_E547insQTS | Insertion |
| Q546H | Missense |
| Q546K | missense |
| Q546L | missense |
| Q546P | missense |
| Q546R | missense |
| E547K | missense |
| D549H | missense |
| D549N | missense |
| D549Y | missense |
| F550S | missense |
| W552G | missense |
| S553N | missense |
| K567R | missense |
| C604R | missense |
| R617W | missense |
| S629C | missense |
| Q643H | missense |
| H665Q | missense |
| H701R | missense |
| E726K | missense |
| R770Q | missense |
| S773F | missense |
| R777M | missense |
| R818C | missense |
| L866F | Missense |
| S900I | missense |
| C901F | missense |
| C901Y | missense |
| F909L | missense |
| I910M | missense |
| 1910V | missense |
| G914R | missense |
| R916C | missense |
| D926N | missense |
| F930S | missense |
| L938* | Nonsense |
| D939G | missense |
| K944N | missense |
| V952A | missense |
| V955G | missense |
| V955I | missense |
| K966E | missense |
| C971R | missense |
| R975S | missense |
| F977Y | missense |
| E978K | missense |
| R992P | missense |
| N1000D | missense |
| M1004I | missense |
| G1007R | missense |
| A1020V | missense |
| Y1021C | missense |
| Y1021F | missense |
| Y1021H | missense |
| Y1021N | missense |
| R1023L | missense |
| R1023Q | missense |
| T1025A | missense |
| T1025I | missense |
| T1025K | missense |
| T1025N | missense |
| T1025S | missense |
| D1029H | missense |
| D1029Y | missense |
| A1035V | missense |
| E1037K | missense |
| F1039L | missense |
| M1040I | missense |
| M1043I | missense |
| M1043L | missense |
| M1043T | missense |
| M1043L | missense |
| M1043T | missense |
| M1043V | missense |
| N1044K | missense |
| N1044S | missense |
| N1044Y | missense |
| D1045N | missense |
| D1045V | missense |
| A1046E | missense |
| A1046_H1047insTSA | Insertion |
| A1046T | missense |
| A1046V | missense |
| H1047K | missense |
| H1047L | missense |
| H1047P | missense |
| H1047R | missense |
| H1047T | missense |
| H1047X | missense |
| H1047Y | missense |
| H1048L | missense |
| H1048R | missense |
| G1049A | missense |
| G1049C | missense |
| G1049D | missense |
| G1049R | missense |
| G1049S | missense |
| D1056G | Missense |
| H1065L | Missense |
| H1065Y | Missense |
| N1068fs | Frameshift |
| N1068Kfs*5 | frameshift |

In certain example embodiments, the activating mutations of interest are those listed in Table 2:

TABLE 2

| Variant | Impact |
|---|---|
| R38C | Missense |
| R38H | Missense |
| E39K | Missense |
| R88Q | Missense |
| R93P | Missense |
| R93W | Missense |
| E103_G106delinsD | Indel |
| E103_P104del | Deletion |
| P104L | Missense |
| V105_R108del | deletion |
| G106R | Missense |
| G106_R108del | Deletion |
| G106V | Missense |
| R108H | Missense |
| E109_I112delinsD | Indel |
| E110del | Deletion |
| K111del | Deletion |
| K111E | Missense |
| K111N | Missense |
| R115L | Missense |
| G118D | Missense |
| P124L | Missense |
| V344G | Missense |
| V344M | Missense |
| N345I | Missense |
| N345K | Missense |
| N345T | Missense |
| D350G | Missense |
| D350N | Missense |
| G364R | Missense |
| E365K | Missense |
| P366R | Missense |
| C378R | Missense |
| C420R | Missense |
| P447_L455del | Deletion |
| P449_L455del | Deletion |
| P449_N457del | Deletion |
| P449T | Missense |
| H450_P458del | Deletion |
| G451R | Missense |
| E453A | Missense |
| E453K | Missense |
| E453Q | Missense |
| E469delinsDK | Indel |
| P471L | Missense |
| P539R | Missense |
| E542A | Missense |
| E545A | Missense |
| E545D | Missense |
| E545G | Missense |
| E545K | Missense |
| E545Q | Missense |
| Q546E | Missense |
| Q546K | Missense |
| Q546L | Missense |
| Q546P | Missense |
| D549N | Missense |
| C604R | Missense |
| S629C | Missense |
| E726K | Missense |
| C901F | Missense |
| F930S | Missense |
| L938* | Nonsense |
| D939G | Missense |
| K944N | Missense |
| V952A | Missense |
| V955G | Missense |
| V955I | Missense |
| K966E | Missense |
| C971R | Missense |
| G1007R | Missense |
| T1025A | Missense |
| T1025N | Missense |
| T1025S | Missense |
| D1029Y | Missense |
| E1037K | Missense |
| M1043I | Missense |
| M1043L | Missense |
| M1043V | Missense |
| N1044K | Missense |
| H1047L | Missense |
| H1047R | Missense |
| H1047Y | Missense |
| G1049R | Missense |
| N1068fs | Frameshift |
| N1068Kfs*5 | frameshift |

In certain example embodiments, the activating mutation is selected from N345D, H1047R, and E545K.

Accordingly, in a first example embodiment, the present invention is a method of treating a subject suffering from a cancer. The method comprises the steps of: obtaining a sample of a cancer cell from the subject; determining a sequence of a phosphatidylinositol 3-kinase catalytic subunit (PIK3CA) protein in the sample; and administering a first amount of a DKK1 inhibitor to the subject determined to have the sequence of PIK3 CA protein (SEQ ID NO:23) that includes an activating mutation. The cancer can be an epithelial endometrial cancer or an epithelial ovarian cancer.

In a second example embodiment, the present invention is a method of treating a cancer in a subject in need thereof. The method comprises administering a first amount of a DKK1 inhibitor to the subject, wherein the subject is determined to have an activating mutation of a phosphatidylinositol 3-kinase catalytic subunit (PIK3CA) protein (SEQ ID NO:23). The cancer can be an epithelial endometrial cancer or an epithelial ovarian cancer.

In a first aspect of the first and second example embodiments, the DKK1 inhibitor is a DKK1 antibody or antigen binding-fragment thereof.

In a second aspect of the first and second example embodiments, the DKK1 antibody, or antigen binding-fragment thereof comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, LCDR2 has the amino sequence of SEQ ID NO:2, LCDR3 has the amino sequence of SEQ ID NO:3, HCDR1 has the amino sequence of SEQ ID NO:4, HCDR2 has the amino sequence of SEQ ID NO:5, and an HCDR3 has the amino sequence of SEQ ID NO:6.

In a third aspect of the first and second example embodiments, the LCVR comprises the amino acid sequence of SEQ ID NO:7 and the HCVR comprises the amino acid sequence of SEQ ID NO:8.

In a fourth aspect of the first and second example embodiments, the LCVR and HCVR comprise amino acid sequences selected from the group consisting of: (i) a LCVR comprising the amino acid sequence of SEQ ID NO:9 and a HCVR comprising the amino acid sequence of SEQ ID NO:10; (ii) a LCVR comprising the amino acid sequence of SEQ ID NO:11 and a HCVR comprising the amino acid sequence of SEQ ID NO:12; (iii) a LCVR comprising the amino acid sequence of SEQ ID NO:13 and a HCVR comprising the amino acid sequence of SEQ ID NO:10; (iv) a LCVR comprising the amino acid sequence of SEQ ID NO:14 and a HCVR comprising the amino acid sequence of SEQ ID NO:10.

In a fifth aspect of the first and second example embodiments, the LCVR comprises the amino acid sequence of SEQ ID NO:11 and the HCVR comprises the amino acid sequence of SEQ ID NO:12.

In a sixth aspect of the first and second example embodiments, the DKK1 antibody comprises a heavy chain and a light chain amino acid sequence selected from the group consisting of a) a heavy chain comprising the amino acid sequence of SEQ ID NO:19 and light chain comprising the amino acid sequence of SEQ ID NO:16, b) a heavy chain comprising the amino acid sequence of SEQ ID NO:17 and a light chain comprising the amino acid sequence of SEQ ID NO:18, c) a heavy chain comprising the amino acid sequence of SEQ ID NO:19 and a light chain comprising the amino acid sequence of SEQ ID NO:20, and d) a heavy chain comprising the amino acid sequence of SEQ ID NO:19 and a light chain comprising the amino acid sequence of SEQ ID NO:21.

In a seventh aspect of the first and second example embodiments, the DKK1 antibody comprises a heavy chain comprising the amino acid sequence of SEQ ID NO:17 and a light chain comprising the amino acid sequence of SEQ ID NO:18.

In an eighth aspect of the first and second example embodiments, the subject is a human.

In a ninth aspect of the first and second example embodiments, the method further comprises administering to the subject a second amount of a second therapeutic agent.

Examples of the second therapeutic agent include an anti-PD-1/PD-L1 monoclonal antibody or antigen binding-fragment thereof, such as an anti-PD-1/PD-L1 monoclonal antibody pembrolizumab. Further examples of second therapeutic agents include taxanes, cisplatin, and gemcitabine.

As used herein, the term “taxanes” includes paclitaxel, docetaxel, carbazitaxel, and their derivatives that possess antineoplastic properties. For example, “paclitaxel” includes both naturally derived and chemically synthesized paclitaxel. Paclitaxel is sold as TAXOL®. Derivatized paclitaxels suitable for use in the invention described herein include deoxygenated paclitaxel compounds such as those described in U.S. Pat. No. 5,440,056, albumin-bound paclitaxel (ABRAXANE), DHA-paclitaxel, and PG-paclitaxel. Chemical formulas for paclitaxel and derivatives thereof are known and described in the art. Other taxane compounds are disclosed in “Synthesis and Anticancer Activity of Taxol other Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elvesier, Amsterdam 1986), pp. 219-235. See also, for example, U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506. The term “docetaxel” includes both naturally derived and chemically synthesized compounds docetaxel. Docetaxel is sold as TAXOTERE®.

In a tenth aspect of the first and second embodiments, the method further comprises administering to the subject a second amount of a second therapeutic agent, wherein the second agent is a paclitaxel.

In an eleventh aspect of the first and second embodiments, the method further comprises administering to the subject a second amount of a second therapeutic agent, wherein the second therapeutic agent is pembrolizumab.

In a twelfth aspect of the first and second embodiments, the method further comprises administering to the subject a second amount of a second therapeutic agent, wherein the DKK1 antagonist is the DKN01 antibody, and the second therapeutic agent is paclitaxel.

In a thirteenth aspect of the first and second embodiments, the method further comprises administering to the subject a second amount of a second therapeutic agent, and a third amount of a third therapeutic agent.

In a fourteenth aspect of the first and second embodiments, the method further comprises administering to the subject a second amount of a second therapeutic agent and a third amount of a third therapeutic agent, wherein the second therapeutic agent is gemcitabine and the third therapeutic agent is a cisplatin.

In a fifteenth aspect of the first and second embodiments, the mutation is at least one of N345D, H1047R, and E545K.

In a sixteenth aspect of the first and second embodiments, the mutation is any one of the mutations of amino acid residues listed in Table 1.

### Dkk-1 Antibody

Dkk-1 antibodies have been described previously (see, e.g., U.S. Pat. Nos. 8,148,498 and 7,446,181, incorporated by reference herein in their entireties). The Dkk-1 antibody or antigen-binding fragment thereof disclosed herein relates to human engineered antibodies that bind to a human Dkk-1 comprising the amino acid sequence set for in SEQ ID NO: 27, or fragments thereof. The present Dkk-1 antibodies are therapeutically useful Dkk-1 antagonists possessing a number of desirable properties. For example, the Dkk-1 antibodies block Dkk-1 mediated inhibition of alkaline phosphatase, a marker or osteoblast activity, as well as treat various types of cancer (e.g., non-small cell lung cancer).

A full-length antibody as it exists naturally is an immunoglobulin molecule comprising 2 heavy (H) chains and 2 light (L) chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (“FR”). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions is in accordance with the well-known Kabat numbering convention.

Light chains are classified as kappa or lambda, and are characterized by a particular constant region as known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

As used herein, the term “monoclonal antibody” (Mab) refers to an antibody that is derived from a single copy or clone including, for example, any eukaryotic, prokaryotic, or phage clone, and not the method by which it is produced. Mabs of the present invention preferably exist in a homogeneous or substantially homogeneous population. Complete Mabs contain 2 heavy chains and 2 light chains.

Unless specified otherwise, the term “Dkk-1 antibody” encompasses both a full-length antibody as well as an antigen binding-fragment of the Dkk-1 antibody.

“Antigen-binding fragments” of such monoclonal antibodies include, for example, Fab fragments, Fab′ fragments, F(ab′)₂ fragments, and single chain Fv fragments. Monoclonal antibodies and antigen-binding fragments thereof can be produced, for example, by recombinant technologies, phage display technologies, synthetic technologies, e.g., CDR-grafting, or combinations of such technologies, or other technologies known in the art. For example, mice can be immunized with human DKK-1 or fragments thereof, the resulting antibodies can be recovered and purified, and determination of whether they possess binding and functional properties similar to or the same as the antibody compounds disclosed herein can be assessed by the methods known in the art. Antigen-binding fragments can also be prepared by conventional methods. Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15, ISBN 0-87969-314-2.

Monoclonal Dkk-1 antibodies disclosed herein are engineered to comprise framework regions that are substantially human or fully human surrounding CDRs derived from a non-human antibody. “Antigen-binding fragments” of such human engineered antibodies include, for example, Fab fragments, Fab′ fragments, F(ab′)₂ fragments, and single chain Fv fragments. “Framework region” or “framework sequence” refers to any one of framework regions 1 to 4. Human engineered antibodies and antigen-binding fragments thereof encompassed by the antibodies disclosed herein include molecules wherein any one or more of framework regions 1 to 4 is substantially or fully human, i.e., wherein any of the possible combinations of individual substantially or fully human framework regions 1 to 4, is present. For example, this includes molecules in which framework region 1 and framework region 2, framework region 1 and framework region 3, framework region 1, 2, and 3, etc., are substantially or fully human. Substantially human frameworks are those that have at least about 80% sequence identity to a known human germline framework sequence. Preferably, the substantially human frameworks have at least about 85%, about 90%, about 95%, or about 99% sequence identity to a known human germline framework sequence.

Human engineered antibodies in addition to those disclosed herein exhibiting similar functional properties can be generated using several different methods. The specific antibody compounds disclosed herein can be used as templates or parent antibody compounds to prepare additional antibody compounds. In one approach, the parent antibody compound CDRs are grafted into a human framework that has a high sequence identity with the parent antibody compound framework. The sequence identity of the new framework will generally be at least about 80%, at least about 85%, at least about 90%, at least about 95%, or at least about 99% identical to the sequence of the corresponding framework in the parent antibody compound. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful in humanizing mouse antibodies include U.S. Pat. Nos. 4,816,397; 5,225,539, and 5,693,761; computer programs ABMOD and ENCAD as described in Levitt (1983) J Mol. Biol. 168:595-620; and the method of Winter and co-workers (Jones et al. (1986) Nature 321:522-525; Riechmann et al. (1988) Nature 332:323-327; and Verhoeyen et al. (1988) Science 239:1534-1536). Methods for identifying residues to consider for back-mutation are known in the art (see, e.g., U.S. Pat. No. 8,148,498).

The DKK1 antibody administered in the method of treatment described herein comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, HCDR1 has the amino sequence of SEQ ID NO:4, and HCDR2 has the amino sequence of SEQ ID NO:5.

In one embodiment, the DKK1 antibody comprises a LCDR1 having the amino sequence of SEQ ID NO:1, LCDR2 having the amino sequence of SEQ ID NO:2, LCDR3 having the amino sequence of SEQ ID NO:3, HCDR1 having the amino sequence of SEQ ID NO:4, HCDR2 having the amino sequence of SEQ ID NO:5, and HCDR3 having the amino sequence of SEQ ID NO:6.

In another embodiment, the DKK1 antibody comprises a LCVR having the amino acid sequence of SEQ ID NO:7 and a HCVR having the amino acid sequence of SEQ ID NO:8. In a particular embodiment, the LCVR comprises the amino acid sequence of SEQ ID NO:11 and the HCVR comprises the amino acid sequence of SEQ ID NO:12.

In further embodiments, the DKK1 antibody comprises a heavy chain (HC) having the amino acid sequence of SEQ ID NO:17 and a light chain (LC) having the amino acid sequence of SEQ ID NO:18. The DKK1 antibody or antigen binding-fragment thereof comprising the HC and LC amino acid sequence of SEQ ID NO:17 and SEQ ID NO:18, respectively, is referred to herein as DKN-01. In particular, DKN-01 has the molecular/empirical formula C₆₃₉₄ H₉₈₁₀ N₁₆₉₈ O₂₀₁₂ S42 and a molecular weight of 144015 Daltons (intact).

In certain embodiments, the DKK1 antibody disclosed herein is an IgG₄ antibody with a neutralizing activity against human DKK1 comprising the sequence set forth in SEQ ID NO: 22, of a fragment thereof. For example, canonical Wnt signaling is important for osteoblast differentiation and activity. Wnt-3a combined with BMP-4 induces multipotent mouse C2C12 cells to differentiate into osteoblasts with a measurable endpoint of alkaline phosphatase (“AP”), a marker of osteoblast activity. DKK1, an inhibitor of canonical Wnt signaling, inhibits the differentiation and production of AP. Neutralizing DKK1 antibodies prevent DKK1-mediated inhibition of AP. Antibodies which block DKK1 inhibitory activity prevent the loss of AP activity (see U.S. Pat. No. 8,148,498). In a particular embodiment, the DKK1 antibody possessing neutralizing activity is DKN-01, which is an IgG₄ antibody.

The DKK1 antibodies disclosed herein possess high affinity (Kd) to DKK1 (e.g., human DKK1, SEQ ID NO:22), as described in U.S. Pat. No. 8,148,498. For example, the present DKK1 antibodies possess a Kd of between 0.5×10⁻¹² M and 3.0×10⁻¹¹ M, at 37° C.

### Modes of Administration

The DKK1 antibody and other therapeutics agents used in combination with the DKK1 antibody (e.g., pembrolizumab, paclitaxel, cisplatin, gemcitabine etc.) for use in the methods or compositions of the invention can be formulated for parenteral, oral, transdermal, sublingual, buccal, rectal, intranasal, intrabronchial or intrapulmonary administration.

For parenteral administration, the compounds for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For oral administration the compounds can be of the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the methods or compositions of the invention can be in the form of tablets or lozenges formulated in a conventional manner.

For rectal administration, the compounds for use in the methods or compositions of the invention can be in the form of suppositories.

For sublingual administration, tablets can be formulated in conventional manner.

For intranasal, intrabronchial or intrapulmonary administration, conventional formulations can be employed.

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use in the method of the invention can be administered in the form of microparticles, for example, by injection or in the form of wafers or discs by implantation. Various methods of formulating controlled release drug preparations are known in the art.

Administration of a compound (e.g., the DKK1 antibody alone or in combination with one or more additional therapeutic agent), or pharmaceutically acceptable salt thereof, or a composition comprising one or more compound (or pharmaceutical salt thereof) of the invention useful to practice the methods described herein, can be continuous, hourly, four times daily, three time daily, twice daily, once daily, once every other day, twice weekly, once weekly, once every two weeks, once a month, or once every two months, or longer, or some other intermittent dosing regimen.

Examples of administration of a compound, or a composition comprising one or more compound (or pharmaceutical salt thereof) of the invention include peripheral administration. Examples of peripheral administration include oral, subcutaneous, intraperitoneal, intramuscular, intravenous, rectal, transdermal, or intranasal forms of administration.

As used herein, peripheral administration includes all forms of administration of a compound or a composition comprising a compound of the instant invention which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

### Combination Therapy

The DKK1 antibody and one or more second therapeutic agents (e.g., pembrolizumab, paclitaxel, cisplatin, gemcitabine etc.) for use in the methods or compositions of the invention can be formulated separately or in combination for parenteral, oral, transdermal, sublingual, buccal, rectal, intranasal, intrabronchial or intrapulmonary administration.

The DKK1 antibody disclosed herein can be used for treating gynecological cancer (e.g. an epithelial endometrial cancer or an epithelial ovarian cancer) in combination with pembrolizumab. Such combination administration can be by means of a single dosage form which includes a DKK1 antibody and pembrolizumab, such single dosage form including a tablet, capsule, spray, inhalation powder, injectable liquid or the like. Combination administration can comprise a further additional agent (e.g., chemotherapeutic agent) in addition to the single dosage form. Alternatively, combination administration can be by means of administration of two different dosage forms, with one dosage form containing a DKK1 antibody, and the other dosage form including a second amount of pembrolizumab. In this instance, the dosage forms may be the same or different. Without wishing to limit combination therapies, the following exemplifies certain combination therapies which may be employed. It is understood that additional chemotherapeutic agents beyond the required second amount of pembrolizumab can be employed in the method described herein.

The second amount of pembrolizumab can be administered before, simultaneously with, or after the administration of a DKK1 antibody. Accordingly, a DKK1 antibody and pembrolizumab can be administered together in a single formulation or can be administered in separate formulations, e.g., either simultaneously or sequentially, or both. For example, if a DKK1 antibody and pembrolizumab are administered sequentially in separate compositions, the DKK1 antibody can be administered before or after pembrolizumab. The duration of time between the administration of a DKK1 antibody and the second amount of pembrolizumab will be easily determined by a person of ordinary skill in the art. In certain embodiments, the DKK1 antibody can precede or follow pembrolizumab immediately, or after some duration of time deemed to be appropriate by a skilled practitioner.

In addition, the DKK1 antibody and the second amount of pembrolizumab may or may not be administered on similar dosing schedules. For example, the DKK1 antibody and pembrolizumab may have different half-lives and/or act on different time-scales such that the DKK1 antibody is administered with greater frequency than pembrolizumab or vice-versa. For example, the DKK1 antibody and pembrolizumab can be administered together (e.g., in a single dosage or sequentially) on one day, followed by administration of only the chemotherapeutic agent (or a different chemotherapeutic) a set number of days later. The number of days in between administration of therapeutic agents can be appropriately determined according to the safety, pharmacokinetics and pharmacodynamics of each drug. Either the DKK1 antibody or pembrolizumab can be administered acutely or chronically.

In a particular embodiment, the treatment period for the combination treatment of DKN-01 and pembrolizumab is a 21-Day cycle which can be repeated until the patient is determined to not be gaining any clinical benefit from the combination therapy. For example, the patient can undergo from about one cycle to about 30 cycles of treatment (e.g., 1, 2, 3, 4, 5, 6, 7, 8, 9, 10, 11, 12, 13, 14, 15, 16, 7, 18, 19, 20, 21, 22, 23, 24, 25, 26, 27, 28, 29, 30). In another embodiment, the subject is being treated for a gynecological cancer. Treatment comprises a combined administration of a DKK1 antibody, such as DKN01, and paclitaxel, following the clinical trials described herein.

As used herein, an “effective amount” refers to an amount of a therapeutic agent or a combination of therapeutic agents that is therapeutically or prophylactically sufficient to treat the target disorder. An effective amount will depend on the age, gender, and weight of the patient, the current medical condition of the patient, and the nature of the gynecological cancer being treated. Those of skill in the art will be able to determine appropriate dosages depending on these and other factors.

In an example embodiment, a subject in need thereof receives a monotherapy (i.e. is being administered a first amount of a first therapeutic agent), so that the first amount of the first therapeutic agent is an effective amount. In another example embodiment, a subject in need thereof receives a combination therapy, e.g. is being administered a first amount of a first therapeutic agent and a second amount of a second therapeutic agent, so that the first amount and the second amount, in combination, is an effective amount. In further embodiment, a combination therapy can employ a third amount of a third therapeutic agent, so that the first amount, the second amount, and the third amount, in combination, is an effective amount.

An effective amount can be achieved in the methods or compositions of the invention by coadministering a first amount of a DKK1 antibody (or a pharmaceutically acceptable salt, hydrate or solvate thereof) and a second amount of pembrolizumab. In one embodiment, the DKK1 antibody and pembrolizumab are each administered in a respective effective amount (e.g., each in an amount which would be therapeutically effective if administered alone). In another embodiment, the DKK1 antibody and pembrolizumab each is administered in an amount that, alone, does not provide a therapeutic effect (a sub-therapeutic dose). In yet another embodiment, the DKK1 antibody can be administered in an effective amount, while pembrolizumab is administered in a sub-therapeutic dose. In still another embodiment, the DKK1 antibody can be administered in a sub-therapeutic dose, while pembrolizumab is administered in an effective amount.

Suitable doses per administration for a DKK1 antibody include doses of about or greater than about 15 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, or about 3,000 mg. Each suitable dose can be administered over a period time deemed appropriate by a skilled practitioner. For example, each suitable dose can be administered over a period of about 30 minutes and up to about 1 hour, about 2 hours, about 3, hours, about 4 hours, about 5 hours, about 6 hours, about 7 hours, or about 8 hours. In a specific embodiment, a suitable does for the DKK1 antibody (e.g., DKN-01) can from about 50 mg to about 300 mg (such as 50 mg, 75 mg, 100 mg, 125 mg, 150 mg, 175 mg, 200 mg, 225 mg, 250 mg, 275 mg or 300 mg). The selected dose can be administered intravenously over a period of about 30 minutes to about 2 hours. In a particular embodiment, a suitable dose for DKK1 antibody can be about 150 mg administered over a period of about 30 minutes and up to about 2 hours. Another suitable dose for the DKK1 antibody can be about 300 mg administered over a period of about 30 minutes and up to about 2 hours. Administration of these doses over the recited period of time can be accomplished using an intravenous route.

Suitable doses per administration for pembrolizumab can be determined based on the recommended dosing found on the label. For example, a suitable dose per administration of pembrolizumab is from about 50 mg to about 200 mg intravenously over at least a 30 minute period. This administration can be repeated every three weeks. In a particular embodiment, a suitable dose per administration is about 200 mg over a 30 minute infusion period using an intravenous route. This dose can be repeated every three weeks. Other suitable doses of pembrolizumab include 2 mg/kg Q3W (every three weeks), 10 mg/kg Q3W (every three weeks), and 10 mg/kg Q2W (every two weeks). In a particular embodiment, the dose of pembrolixumab is 200 mg intravenously. In one aspect, the 200 mg is administered over 30 minutes.

Suitable doses per administration for taxanes (e.g., paclitaxel) can be determined based on the recommended dosing found on the label. For example, a suitable dose per administration of paclitaxel is from about 200 mg/m2 to about 20 mg/m². In a particular embodiment, the dose of paclitaxel is 80 mg/m². The taxane (e.g., paclitaxel) can be administered intravenously. Intravenous administration can be over about one hour.

Suitable doses per administration for gemcitabine can be determined based on the recommended dosing found on the label. For example, a suitable dose per administration of gemcitabine is from about 2000 mg/m² to about 500 mg/m². In a particular embodiment, the dose of gemcitabine is 1000 mg/m².

Suitable doses per administration for cisplatin can be determined based on the recommended dosing found on the label. For example, a suitable dose per administration of cisplatin is from about 10 mg/m² to about 40 mg/m². In a particular embodiment, the dose of cisplatin is 20 mg/m².

As used herein, the term “subject” refers to a mammal, preferably a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like). The terms “subject” and “patient” are used interchangeably herein. In a particular embodiment, the subject has been undergone at least one (e.g., 1, 2, 3, 4 or 5 prior treatments) prior treatment therapy for the cancer being treated. Such therapies include chemotherapy (e.g., carboplatin, paclitaxel), radiation therapy, surgery to remove the cancer. A prior treatment therapy can include chemotherapy alone (with one or more drugs), radiation alone, surgery alone or any combination of the three. For example, A prior treatment therapy can include a combination of radiation and chemotherapy or a combination of surgery and radiation or a combination of surgery, radiation and chemotherapy. In some instances, the subject's disease is refractory to such prior treatment.

As used herein “treating” includes achieving, partially or substantially, delaying, inhibiting or preventing the progression of clinical indications related to the gynecological cancer. For example, “treating” includes reduction in tumor growth, or prevention of further growth, as detected by standard imaging methods known in the art, including, for example, computed tomography (CT) scan, magnetic resonance imaging (MRI), chest x-ray, and CT/positron emission tomography (CT/PET) scans, and evaluated according to guidelines and methods known in the art. For example, responses to treatment can be evaluated through the Response Evaluation Criteria in Solid Tumors (RECIST) (Revised RECIST Guideline version 1.1; see Eisenhauer et al., Eur. J. Cancer 45(2):228-47, 2009). Thus, in some embodiments, “treating” refers to a Complete Response (CR), which is defined according to the RECIST guideline as the disappearance of all target lesions, or a Partial Response (PR), which is defined as at least a 30% decrease in the sum of diameter of target lesions, taking as reference the baseline sum diameters. Other means for evaluating tumor response to treatment include evaluation of tumor markers and evaluation of performance status (e.g., assessment of creatinine clearance; see Cockcroft and Gault, Nephron. 16:31-41, 1976).

### Pharmaceutical Composition

The Dkk-1 antibody and chemotherapeutic agents disclosed herein can be incorporated into pharmaceutical compositions suitable for administration. Such compositions typically comprise the antibody, or one or more chemotherapeutic agents, or both, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL(™) (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a Dkk-lantibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid-derivatives. Transmucosal administration can be accomplished through the use of nasal sprays or suppositories.

For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### SEQUENCES

The following are sequences of the DKN01 antibody that can be employed in the practice of the various example embodiments described herein.

LCDR1
(SEQ ID NO: 1)
His Ala Ser Asp Ser Ile Ser Asn Ser Leu His
LCDR2
(SEQ ID NO: 2)
Tyr Xaa Arg Gln Ser Xaa Gln
wherein Xaa at position 2 is Gly or Ala;
and Xaa at position 6 is Ile or Glu
LCDR3
(SEQ ID NO: 3)
Gln Gln Ser Xaa Ser Trp Pro Leu His
wherein Xaa at position 4 is Glu or Ala
HCDR1
(SEQ ID NO: 4)
Gly Phe Thr Phe Ser Ser Tyr Thr Met Ser
HCDR2
(SEQ ID NO: 5)
Thr Ile Ser Gly Gly Gly Phe Gly Thr Tyr
Tyr Pro Asp Ser Val Lys
HCDR3
(SEQ ID NO: 6)
Pro Gly Tyr Xaa Asn Tyr Tyr Phe Asp Ile,
wherein Xaa at position 4 is His or Asn
LCVR
(SEQ ID NO: 7)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Xaa Arg Gln Ser Xaa
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Xaa Ser Trp Pro Leu His Phe Gly
Gly Gly Thr Lys Val Glu Ile Lys,
wherein Xaa at position 51 is Gly or Ala;
Xaa at position 55 is Ile or Glu and Xaa
at position 92 is Glu or Ala.
HCVR
(SEQ ID NO: 8)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr Xaa Asn Tyr Tyr Phe Asp Ile Trp Gly
Gln Gly Thr Thr Val Thr Val Ser Ser,
wherein Xaa at position 102 is His or Asn
LCVR
(SEQ ID NO: 9)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Gly Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Glu Ser Trp Pro Leu His Phe Gly
Gly Gly Thr Lys Val Glu Ile Lys
HCVR
(SEQ ID NO: 10)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr His Asn Tyr Tyr Phe Asp Ile Trp Gly
Gln Gly Thr Thr Val Thr Val Ser Ser
LCVR
(SEQ ID NO: 11)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Ala Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Glu Ser Trp Pro Leu His Phe Gly
Gly Gly Thr Lys Val Glu Ile Lys
HCVR
(SEQ ID NO: 12)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr Asn Asn Tyr Tyr Phe Asp Ile Trp Gly
Gln Gly Thr Thr Val Thr Val Ser Ser
LCVR
(SEQ ID NO: 13)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Gly Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Ala Ser Trp Pro Leu His Phe Gly
Gly Gly Thr Lys Val Glu Ile Lys
LCVR
(SEQ ID NO: 14)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Ala Arg Gln Ser Glu
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Ala Ser Trp Pro Leu His Phe Gly
Gly Gly Thr Lys Val Glu Ile Lys
HC
(SEQ ID NO: 15)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr His Asn Tyr Tyr Phe Asp Ile Trp Gly
Gln Gly Thr Thr Val Thr Val Ser Ser Ala Ser
Thr Lys Gly Pro Ser Val Phe Pro Leu Ala Pro
Cys Ser Arg Ser Thr Ser Glu Ser Thr Ala Ala
Leu Gly Cys Leu Val Lys Asp Tyr Phe Pro Glu
Pro Val Thr Val Ser Trp Asn Ser Gly Ala Leu
Thr Ser Gly Val His Thr Phe Pro Ala Val Leu
Gln Ser Ser Gly Leu Tyr Ser Leu Ser Ser Val
Val Thr Val Pro Ser Ser Ser Leu Gly Thr Lys
Thr Tyr Thr Cys Asn Val Asp His Lys Pro Ser
Asn Thr Lys Val Asp Lys Arg Val Glu Ser Lys
Tyr Gly Pro Pro Cys Pro Pro Cys Pro Ala Pro
Glu Ala Ala Gly Gly Pro Ser Val Phe Leu Phe
Pro Pro Lys Pro Lys Asp Thr Leu Met Ile Ser
Arg Thr Pro Glu Val Thr Cys Val Val Val Asp
Val Ser Gln Glu Asp Pro Glu Val Gln Phe Asn
Trp Tyr Val Asp Gly Val Glu Val His Asn Ala
Lys Thr Lys Pro Arg Glu Glu Gln Phe Asn Ser
Thr Tyr Arg Val Val Ser Val Leu Thr Val Leu
His Gln Asp Trp Leu Asn Gly Lys Glu Tyr Lys
Cys Lys Val Ser Asn Lys Gly Leu Pro Ser Ser
Ile Glu Lys Thr Ile Ser Lys Ala Lys Gly Gln
Pro Arg Glu Pro Gln Val Tyr Thr Leu Pro Pro
Ser Gln Glu Glu Met Thr Lys Asn Gln Val Ser
Leu Thr Cys Leu Val Lys Gly Phe Tyr Pro Ser
Asp Ile Ala Val Glu Trp Glu Ser Asn Gly Gln
Pro Glu Asn Asn Tyr Lys Thr Thr Pro Pro Val
Leu Asp Ser Asp Gly Ser Phe Phe Leu Tyr Ser
Arg Leu Thr Val Asp Lys Ser Arg Trp Gln Glu
Gly Asn Val Phe Ser Cys Ser Val Met His Glu
Ala Leu His Asn His Tyr Thr Gln Lys Ser Leu
Ser Leu Ser Leu Gly
LC
(SEQ ID NO: 16)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Gly Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Glu Ser Trp Pro Leu His Phe Gly
Gly Gly Thr Lys Val Glu Ile Lys Arg Thr Val
Ala Ala Pro Ser Val Phe Ile Phe Pro Pro Ser
Asp Glu Gln Leu Lys Ser Gly Thr Ala Ser Val
Val Cys Leu Leu Asn Asn Phe Tyr Pro Arg Glu
Ala Lys Val Gln Trp Lys Val Asp Asn Ala Leu
Gln Ser Gly Asn Ser Gln Glu Ser Val Thr Glu
Gln Asp Ser Lys Asp Ser Thr Tyr Ser Leu Ser
Ser Thr Leu Thr Leu Ser Lys Ala Asp Tyr Glu
Lys His Lys Val Tyr Ala Cys Glu Val Thr His
Gln Gly Leu Ser Ser Pro Val Thr Lys Ser Phe
Asn Arg Gly Glu Cys
HC
(SEQ ID NO: 17)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr Asn Asn Tyr Tyr Phe Asp Ile Trp Gly
Gln Gly Thr Thr Val Thr Val Ser Ser Ala Ser
Thr Lys Gly Pro Ser Val Phe Pro Leu Ala Pro
Cys Ser Arg Ser Thr Ser Glu Ser Thr Ala Ala
Leu Gly Cys Leu Val Lys Asp Tyr Phe Pro Glu
Pro Val Thr Val Ser Trp Asn Ser Gly Ala Leu
Thr Ser Gly Val His Thr Phe Pro Ala Val Leu
Gln Ser Ser Gly Leu Tyr Ser Leu Ser Ser Val
Val Thr Val Pro Ser Ser Ser Leu Gly Thr Lys
Thr Tyr Thr Cys Asn Val Asp His Lys Pro Ser
Asn Thr Lys Val Asp Lys Arg Val Glu Ser Lys
Tyr Gly Pro Pro Cys Pro Pro Cys Pro Ala Pro
Glu Ala Ala Gly Gly Pro Ser Val Phe Leu Phe
Pro Pro Lys Pro Lys Asp Thr Leu Met Ile Ser
Arg Thr Pro Glu Val Thr Cys Val Val Val Asp
Val Ser Gln Glu Asp Pro Glu Val Gln Phe Asn
Trp Tyr Val Asp Gly Val Glu Val His Asn Ala
Lys Thr Lys Pro Arg Glu Glu Gln Phe Asn Ser
Thr Tyr Arg Val Val Ser Val Leu Thr Val Leu
His Gln Asp Trp Leu Asn Gly Lys Glu Tyr Lys
Cys Lys Val Ser Asn Lys Gly Leu Pro Ser Ser
Ile Glu Lys Thr Ile Ser Lys Ala Lys Gly Gln
Pro Arg Glu Pro Gln Val Tyr Thr Leu Pro Pro
Ser Gln Glu Glu Met Thr Lys Asn Gln Val Ser
Leu Thr Cys Leu Val Lys Gly Phe Tyr Pro Ser
Asp Ile Ala Val Glu Trp Glu Ser Asn Gly Gln
Pro Glu Asn Asn Tyr Lys Thr Thr Pro Pro Val
Leu Asp Ser Asp Gly Ser Phe Phe Leu Tyr Ser
Arg Leu Thr Val Asp Lys Ser Arg Trp Gln Glu
Gly Asn Val Phe Ser Cys Ser Val Met His Glu
Ala Leu His Asn His Tyr Thr Gln Lys Ser Leu
Ser Leu Ser Leu Gly
LC
(SEQ ID NO: 18)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Ala Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Glu Ser Trp Pro Leu His Phe Gly
Gly Gly Thr Lys Val Glu Ile Lys Arg Thr Val
Ala Ala Pro Ser Val Phe Ile Phe Pro Pro Ser
Asp Glu Gln Leu Lys Ser Gly Thr Ala Ser Val
Val Cys Leu Leu Asn Asn Phe Tyr Pro Arg Glu
Ala Lys Val Gln Trp Lys Val Asp Asn Ala Leu
Gln Ser Gly Asn Ser Gln Glu Ser Val Thr Glu
Gln Asp Ser Lys Asp Ser Thr Tyr Ser Leu Ser
Ser Thr Leu Thr Leu Ser Lys Ala Asp Tyr Glu
Lys His Lys Val Tyr Ala Cys Glu Val Thr His
Gln Gly Leu Ser Ser Pro Val Thr Lys Ser Phe
Asn Arg Gly Glu Cys
HC
(SEQ ID NO: 19)
Glu Val Gln Leu Val Glu Ser Gly Gly Gly Leu
Val Gln Pro Gly Gly Ser Leu Arg Leu Ser Cys
Ala Ala Ser Gly Phe Thr Phe Ser Ser Tyr Thr
Met Ser Trp Val Arg Gln Ala Pro Gly Lys Gly
Leu Glu Trp Val Ala Thr Ile Ser Gly Gly Gly
Phe Gly Thr Tyr Tyr Pro Asp Ser Val Lys Gly
Arg Phe Thr Ile Ser Arg Asp Asn Ala Lys Asn
Ser Leu Tyr Leu Gln Met Asn Ser Leu Arg Ala
Glu Asp Thr Ala Val Tyr Tyr Cys Ala Arg Pro
Gly Tyr His Asn Tyr Tyr Phe Asp Ile Trp Gly
Gln Gly Thr Thr Val Thr Val Ser Ser Ala Ser
Thr Lys Gly Pro Ser Val Phe Pro Leu Ala Pro
Cys Ser Arg Ser Thr Ser Glu Ser Thr Ala Ala
Leu Gly Cys Leu Val Lys Asp Tyr Phe Pro Glu
Pro Val Thr Val Ser Trp Asn Ser Gly Ala Leu
Thr Ser Gly Val His Thr Phe Pro Ala Val Leu
Gln Ser Ser Gly Leu Tyr Ser Leu Ser Ser Val
Val Thr Val Pro Ser Ser Ser Leu Gly Thr Lys
Thr Tyr Thr Cys Asn Val Asp His Lys Pro Ser
Asn Thr Lys Val Asp Lys Arg Val Glu Ser Lys
Tyr Gly Pro Pro Cys Pro Pro Cys Pro Ala Pro
Glu Ala Ala Gly Gly Pro Ser Val Phe Leu Phe
Pro Pro Lys Pro Lys Asp Thr Leu Met Ile Ser
Arg Thr Pro Glu Val Thr Cys Val Val Val Asp
Val Ser Gln Glu Asp Pro Glu Val Gln Phe Asn
Trp Tyr Val Asp Gly Val Glu Val His Asn Ala
Lys Thr Lys Pro Arg Glu Glu Gln Phe Asn Ser
Thr Tyr Arg Val Val Ser Val Leu Thr Val Leu
His Gln Asp Trp Leu Asn Gly Lys Glu Tyr Lys
Cys Lys Val Ser Asn Lys Gly Leu Pro Ser Ser
Ile Glu Lys Thr Ile Ser Lys Ala Lys Gly Gln
Pro Arg Glu Pro Gln Val Tyr Thr Leu Pro Pro
Ser Gln Glu Glu Met Thr Lys Asn Gln Val Ser
Leu Thr Cys Leu Val Lys Gly Phe Tyr Pro Ser
Asp Ile Ala Val Glu Trp Glu Ser Asn Gly Gln
Pro Glu Asn Asn Tyr Lys Thr Thr Pro Pro Val
Leu Asp Ser Asp Gly Ser Phe Phe Leu Tyr Ser
Arg Leu Thr Val Asp Lys Ser Arg Trp Gln Glu
Gly Asn Val Phe Ser Cys Ser Val Met His Glu
Ala Leu His Asn His Tyr Thr Gln Lys Ser Leu
Ser Leu Ser Leu Gly
LC
(SEQ ID NO: 20)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Gly Arg Gln Ser Ile
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Ala Ser Trp Pro Leu His Phe Gly
Gly Gly Thr Lys Val Glu Ile Lys Arg Thr Val
Ala Ala Pro Ser Val Phe Ile Phe Pro Pro Ser
Asp Glu Gln Leu Lys Ser Gly Thr Ala Ser Val
Val Cys Leu Leu Asn Asn Phe Tyr Pro Arg Glu
Ala Lys Val Gln Trp Lys Val Asp Asn Ala Leu
Gln Ser Gly Asn Ser Gln Glu Ser Val Thr Glu
Gln Asp Ser Lys Asp Ser Thr Tyr Ser Leu Ser
Ser Thr Leu Thr Leu Ser Lys Ala Asp Tyr Glu
Lys His Lys Val Tyr Ala Cys Glu Val Thr His
Gln Gly Leu Ser Ser Pro Val Thr Lys Ser Phe
Asn Arg Gly Glu Cys
LC
(SEQ ID NO: 21)
Glu Ile Val Leu Thr Gln Ser Pro Ala Thr Leu
Ser Leu Ser Pro Gly Glu Arg Ala Thr Leu Ser
Cys His Ala Ser Asp Ser Ile Ser Asn Ser Leu
His Trp Tyr Gln Gln Lys Pro Gly Gln Ala Pro
Arg Leu Leu Ile Tyr Tyr Ala Arg Gln Ser Glu
Gln Gly Ile Pro Ala Arg Phe Ser Gly Ser Gly
Ser Gly Thr Asp Phe Thr Leu Thr Ile Ser Ser
Leu Glu Pro Glu Asp Phe Ala Val Tyr Tyr Cys
Gln Gln Ser Ala Ser Trp Pro Leu His Phe Gly
Gly Gly Thr Lys Val Glu Ile Lys Arg Thr Val
Ala Ala Pro Ser Val Phe Ile Phe Pro Pro Ser
Asp Glu Gln Leu Lys Ser Gly Thr Ala Ser Val
Val Cys Leu Leu Asn Asn Phe Tyr Pro Arg Glu
Ala Lys Val Gln Trp Lys Val Asp Asn Ala Leu
Gln Ser Gly Asn Ser Gln Glu Ser Val Thr Glu
Gln Asp Ser Lys Asp Ser Thr Tyr Ser Leu Ser
Ser Thr Leu Thr Leu Ser Lys Ala Asp Tyr Glu
Lys His Lys Val Tyr Ala Cys Glu Val Thr His
Gln Gly Leu Ser Ser Pro Val Thr Lys Ser Phe
Asn Arg Gly Glu Cys
Human DKK1 Amino Acid Sequence
(SEQ ID NO: 22)
Thr Leu Asn Ser Val Leu Asn Ser Asn Ala Ile
Lys Asn Leu Pro Pro Pro Leu Gly Gly Ala Ala
Gly His Pro Gly Ser Ala Val Ser Ala Ala Pro
Gly Ile Leu Tyr Pro Gly Gly Asn Lys Tyr Gln
Thr Ile Asp Asn Tyr Gln Pro Tyr Pro Cys Ala
Glu Asp Glu Glu Cys Gly Thr Asp Glu Tyr Cys
Ala Ser Pro Thr Arg Gly Gly Asp Ala Gly Val
Gln Ile Cys Leu Ala Cys Arg Lys Arg Arg Lys
Arg Cys Met Arg His Ala Met Cys Cys Pro Gly
Asn Tyr Cys Lys Asn Gly Ile Cys Val Ser Ser
Asp Gln Asn His Phe Arg Gly Glu Ile Glu Glu
Thr Ile Thr Glu Ser Phe Gly Asn Asp His Ser
Thr Leu Asp Gly Tyr Ser Arg Arg Thr Thr Leu
Ser Ser Lys Met Tyr His Thr Lys Gly Gln Glu
Gly Ser Val Cys Leu Arg Ser Ser Asp Cys Ala
Ser Gly Leu Cys Cys Ala Arg His Phe Trp Ser
Lys Ile Cys Lys Pro Val Leu Lys Glu Gly Gln
Val Cys Thr Lys His Arg Arg Lys Gly Ser His
Gly Leu Glu Ile Phe Gln Arg Cys Tyr Cys Gly
Glu Gly Leu Ser Cys Arg Ile Gln Lys Asp His
His Gln Ala Ser Asn Ser Ser Arg Leu His Thr
Cys Gln Arg His

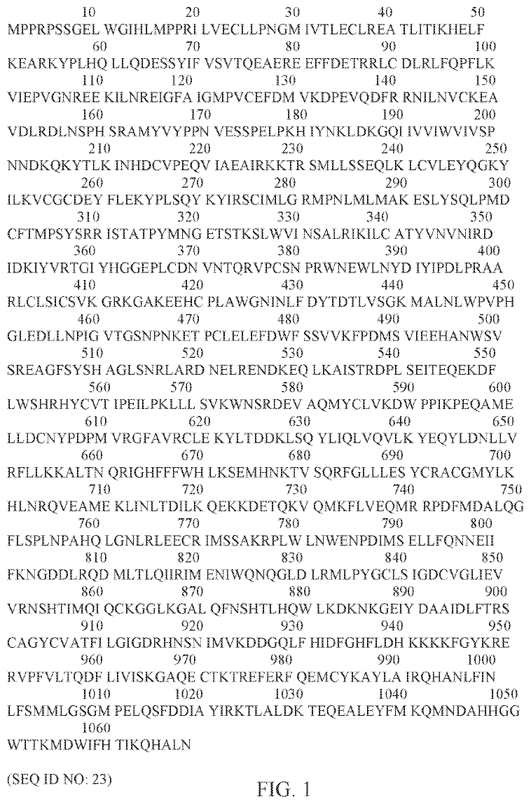

Protein sequence of human PIK3CA (SEQ ID NO:23) is provided in FIG. 1.

### EXEMPLIFICATION

As used herein, “gynecological cancer” refers to cancer of the endometrium (endometrial cancer) and cancer of the ovaries (ovarian cancer). The uterus is lined with a specific tissue called the endometrium. When cancer grows in this lining it is called endometrial cancer. Most cancers of the uterus are endometrial cancers. In certain embodiments, the endometrial cancer is epithelial endometrial cancer (EEC). In another embodiment, the ovarian cancer is epithelial ovarian cancer (EOC). MMMT (malignant mixed Mullerian tumor) is a cancerous growth found in the uterus and ovaries. MMMTs are biphasic, malignant tumors that contain both carcinomatous (malignant epithelial tissue) and sarcomatous (mesenchymal or connective tissue) components. The uterus is the most common site for MMMT. MMMTs are staged like endometrial carcinomas according to the International Federation of Gynecology and Obstetrics and the American Joint Committee on Cancer staging classifications. For purposed of this disclosure, MMMT can be considered a type of EEC or EOC depending upon the organ of origin.

### Example 1

### Summary

In this study, epithelial endometrial cancer (EEC) and epithelial ovarian cancer (EOC) patients were evaluated and it was discovered that the patients with a PIK3CA mutation have improved treatment outcomes when treated with a DKK1-neutralizing therapy.

### Study Design

Subjects: Female patients having Epithelial Endometrial Cancer (EEC) and Epithelial Ovarian Cancer (EOC). Patients with EEC must have a histologically confirmed diagnosis (by either primary surgical specimen or biopsy for recurrence) of recurrent previously treated EEC. Patients with EOC must have a histologically confirmed diagnosis (by either primary surgical specimen or biopsy for recurrence) of recurrent platinum-resistant/refractory EOC, primary peritoneal, or fallopian tube cancer (i.e., disease recurrence within 6 months of completion of or progression during platinum-based chemotherapy).

Treatment regimens:
a) DKN-01 Monotherapy-28 day cycle: 300 mg DKN-01 on day 1 and day 15 of the 28-day cycle. DKN-01 was administered intravenously over a minimum of 30 minutes and up to a maximum of 2 hours.
b) Combination Therapy-DKN-01 and paclitaxel, 28-day cycle: 300 mg of DKN-01 and 80 mg/m² of paclitaxel 300 mg of DKN-01 was administered intravenously over a minimum of 30 minutes and up to a maximum of 2 hours given on day 1 and day 15 of the 28-day cycle. Paclitaxel was administered intravenously over 1 hour on days 1, 8 and 15 of each 28-day cycle according to standard clinical practice. DKN-01 was administered first followed by paclitaxel as separate infusions on day 1 and day 15 of each cycle.

The patient's duration of study participation includes a Screening Period, a Treatment Period and a Follow-up Period. For the Follow-up Period, a visit was scheduled within 30 days after the last treatment administration in the treatment period. After discontinuation of treatment and radiographic documentation of Progressive Disease, all patients will be followed in the survival follow-up phase for survival until death, withdrawal of consent, loss to follow-up, or closure of the study. Survival follow-up will occur 4 times per year (every 3 months) after the end of treatment visit.

Efficacy evaluation:

The primary efficacy endpoint for each study was Objective Response Rate (ORR) as assessed by using the Response Evaluation Criteria in Solid Tumors, version 1.1 (RECIST 1.1) (Eisenhauer EA, Therasse P, Bogaerts J. et al. New response evaluation criteria in solid tumours: revised RECIST guideline (version 1.1). Eur J Cancer. 2009;45(2):228-247. ORR is the best overall response [BOR] of complete response [CR]+partial response [PR]).

Secondary efficacy endpoints in each study were:
a) Objective Disease Control Rate (ODCR) as assessed using RECIST 1.1. ODCR is the CR+PR+stable [SD]>6 weeks (CR=Complete Response and PR=Partial Response);
b) OS (Overall Survival), defined as the time from first study drug dose to death from any cause;
c) PFS (Progression Free Survival), defined as the time from first study drug dose to first radiographically-documented Progressive Disease (PD) as determined using RECIST 1.1 or death due to any cause.
d) TTP, defined as the time from first study drug dose until the date of first radiographically-documented Progressive Disease as determined using RECIST 1.1;
e) DoR (duration of response), defined as the time from initial response (≥PR) until radiographically-documented PD or death; PD is defined using RECIST 1.1;
f) DoCR (duration of complete response), defined as the time from initial CR until radiographically-documented PD or death; PD is defined using RECIST 1.1;
g) DoCB (duration of clinical benefit), defined as the time from the first tumor assessment of CR, PR or SD to the time of PD, as determined using RECIST 1.1, or death due to any cause; and
h) Time to Treatment Failure (TTTF), defined as the time from first study drug dose until the date of discontinuation of DKN-01 for any reason, including PD, toxicity, and death.

### Results

It was discovered that each of the three responding EEC patients (one complete response (CR) and two partial responses (PR)) has a mutation with a known activating impact in the PIK3CA gene. Statistical analysis indicates that there is a significant enrichment in objective response for patients with an activating PIK3CA mutation compared to those that do not have such a mutation (p<0.05); patients with a PIK3CA mutation had a trend for longer median progression-free survival (PFS) and overall survival (OS).

More specifically, the PIK3CA mutations listed in Table 3 were identified:

TABLE 3

| Subject | | | | PIK3CA |
|---|---|---|---|---|
| ID | Type | Treatment | BOR | Mutation |
| 203-008 | EEC | DKN-01 | CR | N345D |
| 222-001 | EEC | DKN-01 | PR | H1047R |
| 223-003 | EEC | DKN-01/Pac | PR | E545K |
| | (MMMT) | | | |
| 213-005 | MMMT | DKN-01 + paclitaxel, | PR | M1043L and |
| | | Paclitaxel discontinued | | amplification |
| | | after 9 cycles; patient | | |
| | | continued on DKN-01 | | |
| | | monotherapy with | | |
| | | residual disease | | |

### Detailed Discussion

PIK3CA mutations and objective response in EEC/EOC patients (N=88). (The number of patients was later increased to 108, the results of the expanded study are shown below in Example 2.) Objective response was noted only in patients with PIK3CA mutation. Specifically, the data is presented in Table 4:

TABLE 4

| Best Overall | PIK3CA | PIK3CA |
|---|---|---|
| Response | Mutation: yes | Mutation: No |
| (N = 88) | (n = 24) | (N = 64) |
| Objective Response | 3 (12.5%) | 0 (0%)* |
| (PR or CR) | | |
| SD | 10 (41.7%) | 28 (43.8%) |
| PD | 8 (33.3%) | 28 (43.8%) |
| NE | 3 (12.5%) | 8 (12.5%) |
| ODCR (PR/CR/SD) | 13 (54.2%) | 28 (43.8%) |

*p-value < 0.05 for comparison of ORR between PIK3CA mutation vs. no PIK3CA mutation by Fisher's exact test (p-value: 0.018) and Chi-squared test after continuity correction (p-value: 0.027).

In Table 4, CR is complete response, PR is partial response, SD is stable disease, PD, is progressive disease, NE is non-evaluable, and ODCR is overall disease control rate.

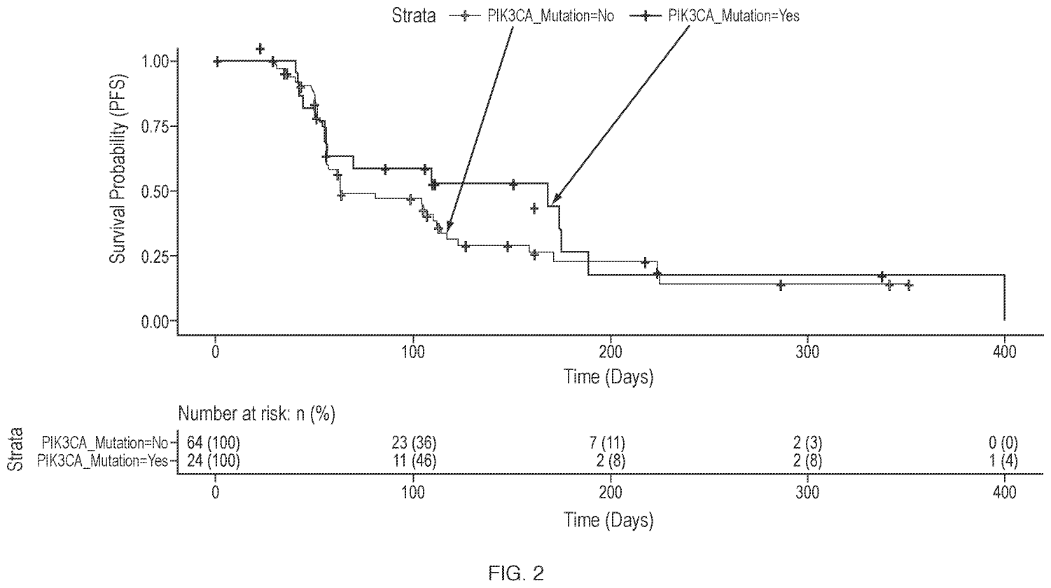

FIG. 2 depicts a plot (Kaplan-Meier (KM) estimates of Progression Free Survival (PFS) probability vs. time), that demonstrates a trend for longer median PFS for the patients having a PIK3CA activating mutation (median: 168 days; 95% CI: 55, 189 days) compared to those who did not have a PIK3CA activating mutation (median: 63 days; 95% CI: 56, 112 days).

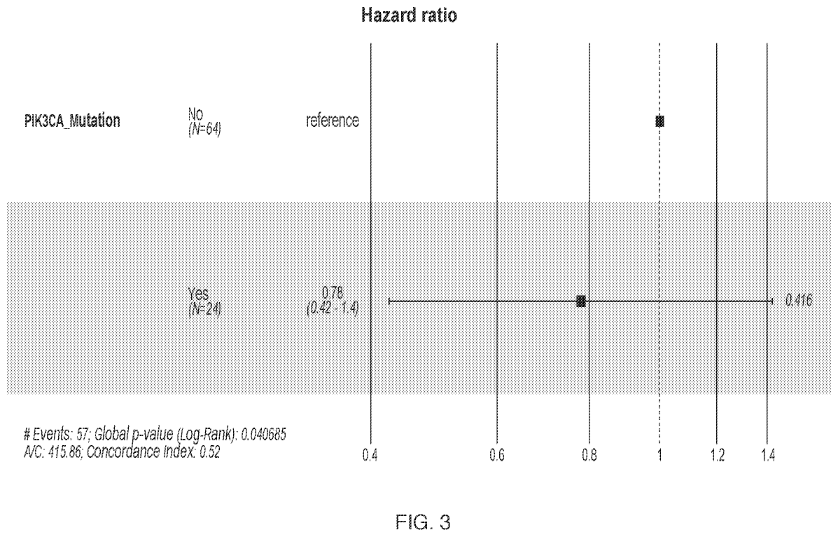

FIG. 3 is a plot showing the hazard ratio (HR, the risk of having an event that is either “radiographic progression” or “dying” from any cause) computed for the pool of 88 EEC/EOC patients based on PFS outcome in patients that have an activating PIK3CA mutation compared to those without activating PIK3CA mutation.

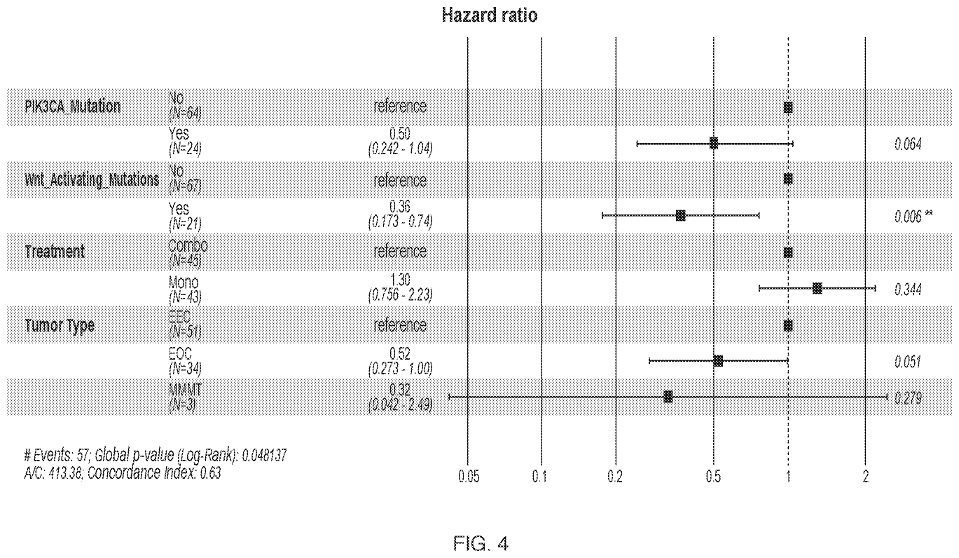

FIG. 4 is a plot showing hazard ratios of the same pool as in FIG. 3, computed for the pool of 88 EEC/EOC patients based on PFS outcome in patients that have an activating PIK3CA mutation compared to those without activating PIK3CA mutation, but adjusted for the presence of a Wnt-pathway activating mutation, treatment modality, and tumor type. Trend for risk reduction for PIK3CA mutation was noted independent of Wnt-pathway activating mutation, treatment modality and tumor type.

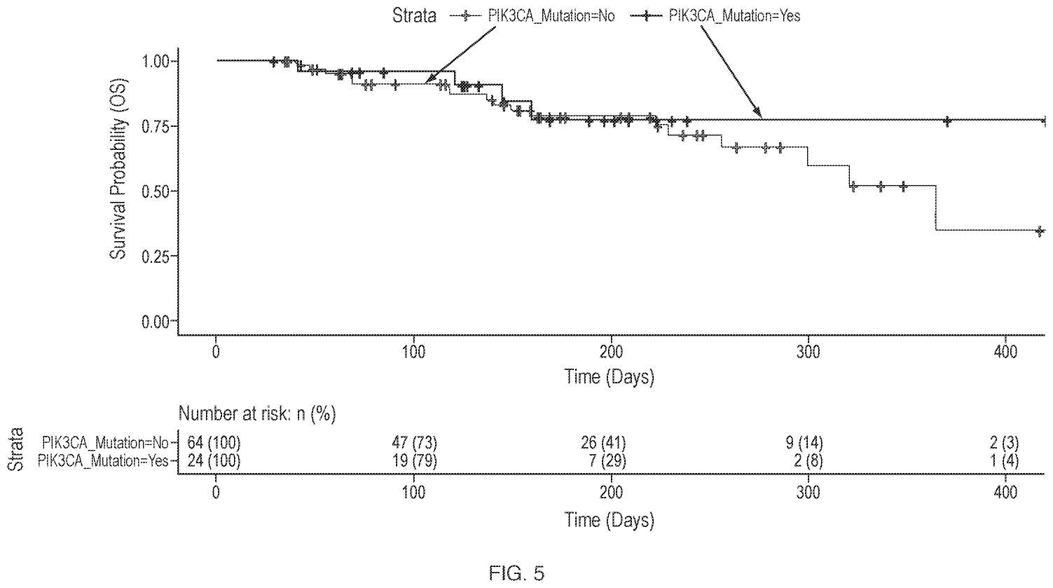

FIG. 5 is depicts a plot (KM estimates of Overall Survival (OS) probability vs. time), that demonstrates a trend for longer median OS for the patients having a PIK3CA activating mutation (median: not reached) compared to those without PIK3CA activating mutation (median: 365 days; 95% CI: 256 days, not reached).

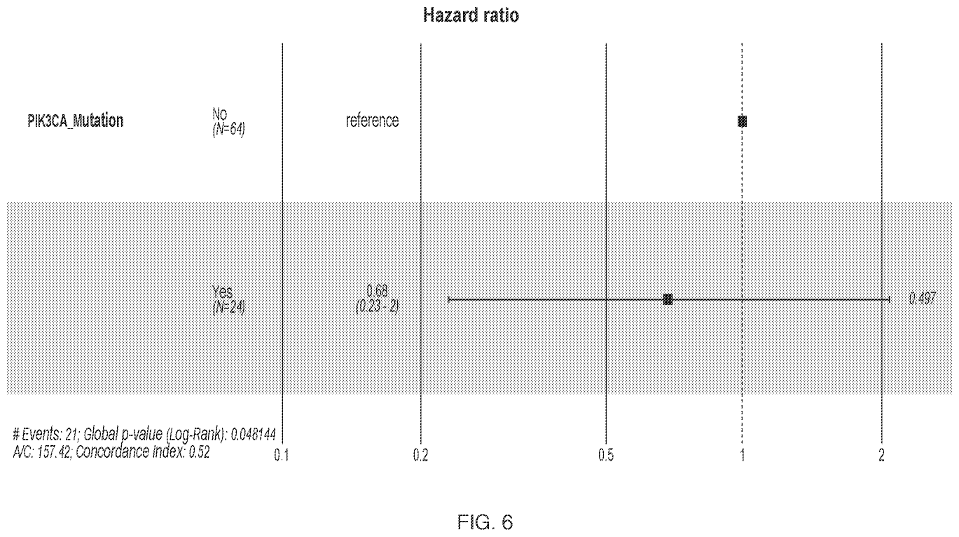

FIG. 6 is a plot showing the hazard ratio computed for the pool of 88 EEC/EOC patients based on the OS outcome in patients that have an activating PIK3CA mutation compared to those without PIK3CA activating mutation.

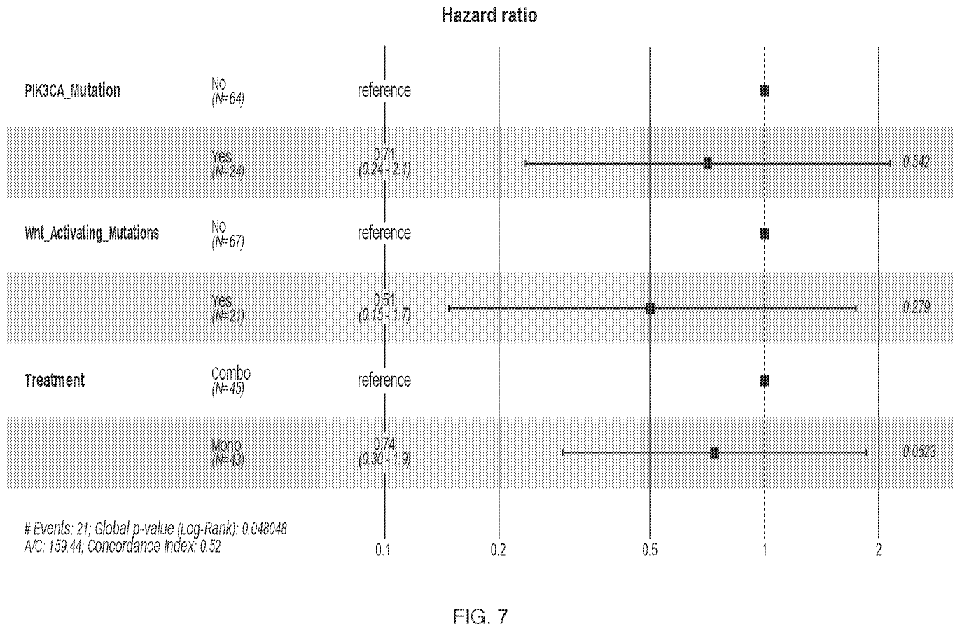

FIG. 7 is a plot showing hazard ratios of the same pool as in FIG. 6, computed for the pool of 88 EEC/EOC patients based on OS outcome in patients that have an activating PIK3CA mutation compared to those without PIK3CA activating mutation, but adjusted for the presence of a Wnt-pathway activating mutation and treatment modality.

The data presented below demonstrates that the predictive value of an activating PIK3CA mutation is independent of Wnt-pathway activating mutations or modality of treatment.

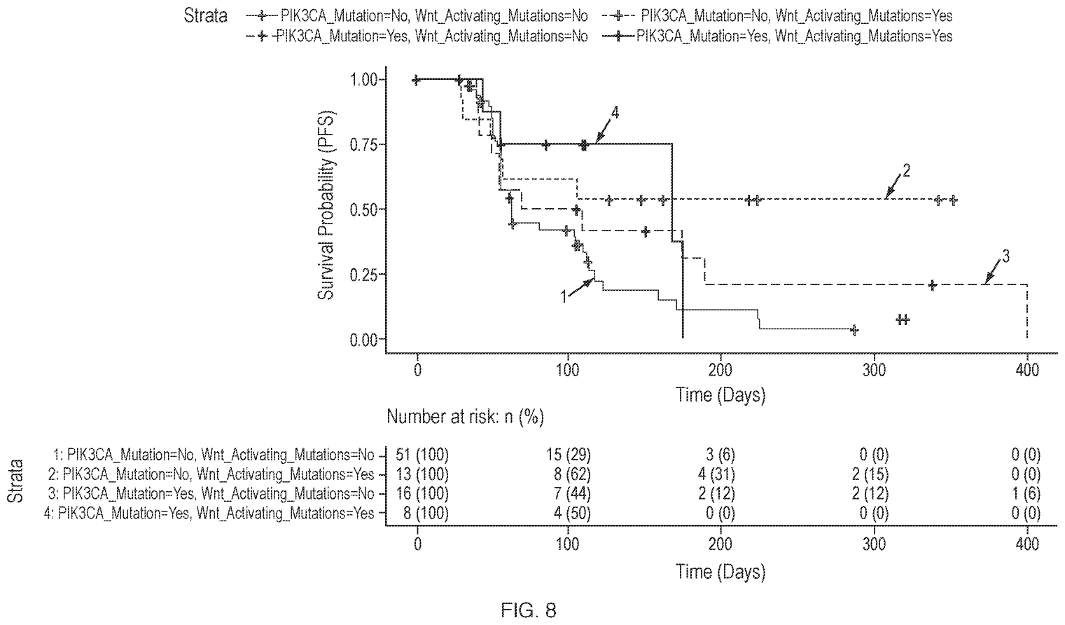

FIG. 8 depicts a plot (KM estimates of Progression Free Survival (PFS) probability vs. time, days post-treatment), that shows PFS probability for the patients having none, either one, or both a PIK3CA activating mutation and a Wnt-pathway activating mutation. Patients with either a Wnt-pathway activating mutation (median: not reached/NR) or both a PIK3CA activating mutation and a Wnt-pathway activating mutation (median: 168 days; 95% CI: 44 days, NR) show a trend for longer median PFS compared to those who do not have a PIK3CA activating mutation and a Wnt-pathway activating mutation (median: 63 days; 95% CI: 56, 110 days).

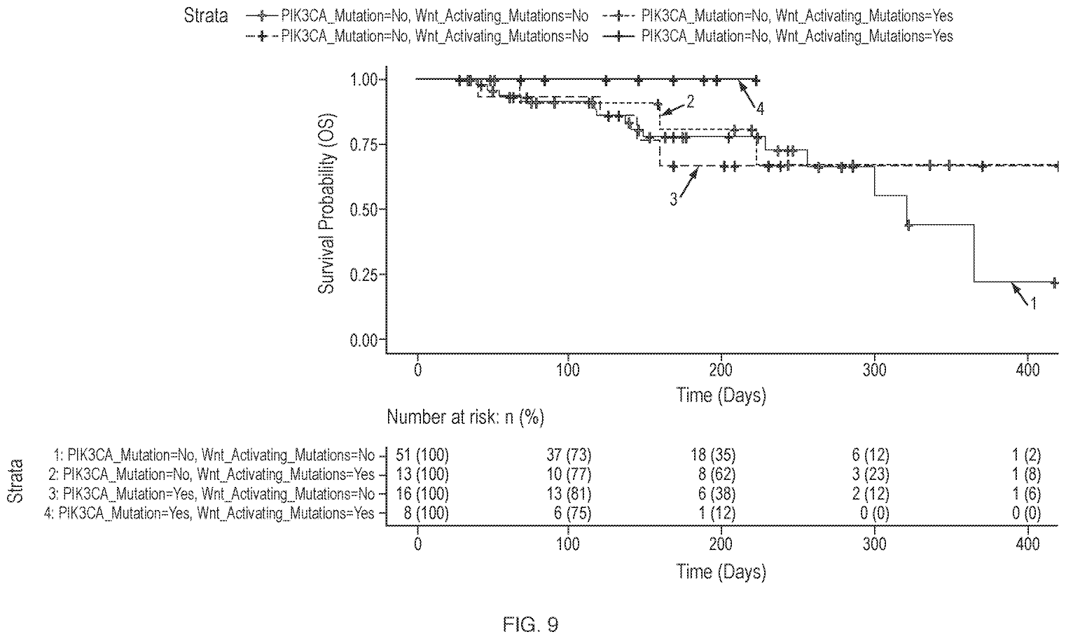

FIG. 9 is depicts a plot (KM estimates of Overall Survival (OS) probability vs. time), that demonstrates higher OS probability for the patients having none, either one, or both a PIK3CA activating mutation and a Wnt-pathway activating mutation. Patients with both a PIK3CA activating mutation and a Wnt-pathway activating mutation show a trend towards longer OS (median: NR; 0 events/8 patients) compared to those who do not have a PIK3CA activating mutation and a Wnt-pathway activating mutation (median: 321 days; 14 events/51 patients).

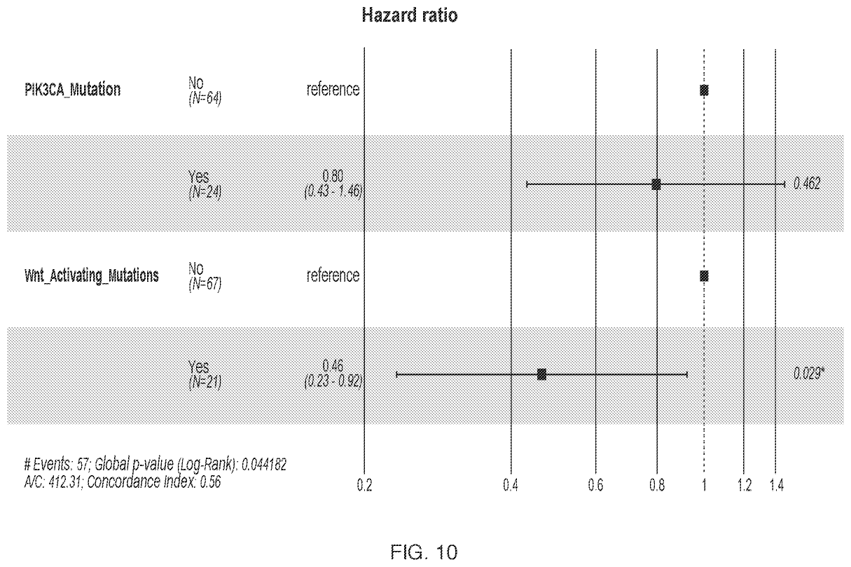

FIG. 10 is a plot showing hazard ratios of the same pool as in FIG. 9, computed for the pool of 88 EEC/EOC patients based on PFS outcome in patients that have an activating PIK3CA mutation compared to those without activating PIK3CA mutation, but adjusted for the presence of a Wnt-pathway activating mutation.

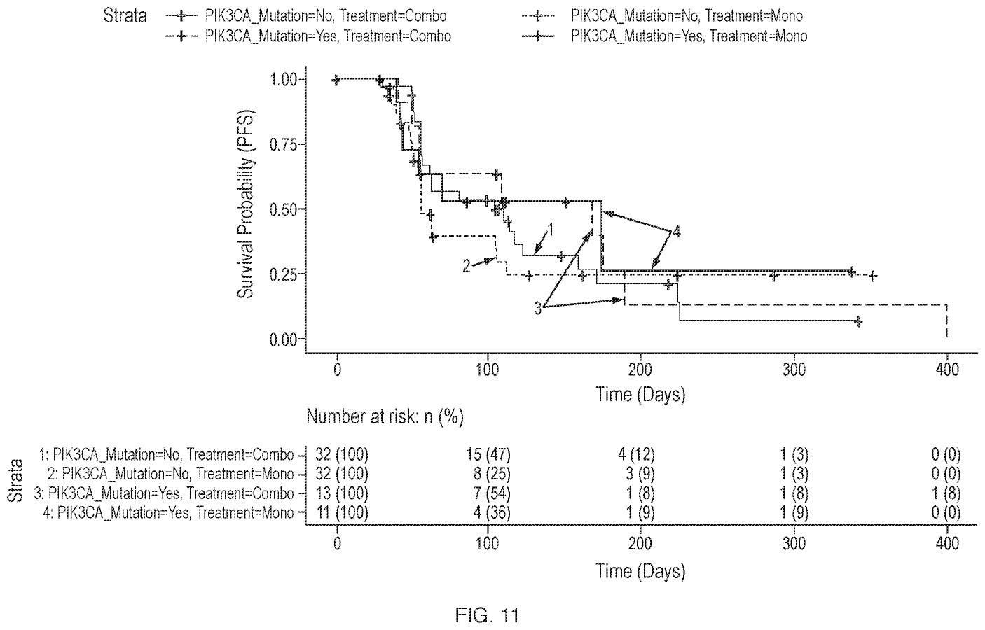

FIG. 11 depicts a plot (KM estimates of Progression Free Survival (PFS) probability vs. time), that shows PFS probability for the patients having a PIK3CA activating mutation and undergoing either a monotherapy or a combination therapy compared to those who did not have a PIK3CA activating mutation. Patients with activating PIK3CA mutation and received either a monotherapy or combination therapy had a trend towards longer median PFS compared to those who did not have a PIK3CA mutation.

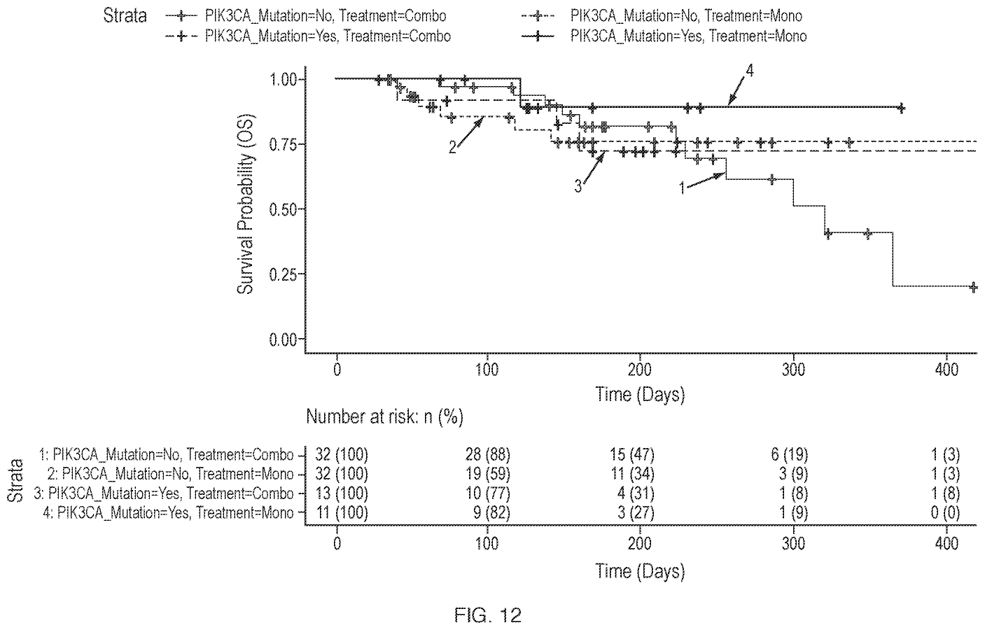

FIG. 12 depicts a plot (KM estimates of Overall Survival (OS) probability vs. time), that shows OS probability for the patients having a PIK3CA activating mutation and undergoing either a monotherapy or a combination therapy compared to those who did not have a PIK3CA activating mutation. Patients with activating PIK3CA mutation and received a monotherapy had a trend towards longer median OS compared to those who did not have a PIK3CA mutation and received monotherapy. Patients with activating PIK3CA mutation and received combination therapy had a trend towards longer median OS compared to those who did not have a PIK3CA mutation and received combination therapy.

### Example 2: Expanded Study of Example 1

Tables 5 through 8 summarize the results of the expanded study outlined in Example 1, where the number of evaluable patients was increased to 108. In Tables 5-8, A confirmed response of CR/PR means that a response of CR/PR is recorded at 1 visit and confirmed by repeat imaging in the next visit when the response was first observed with no evidence of progression between the initial and next visit. In the case where a patient has two consecutive visit responses of PR, as long as there is no PD between PR visits then the patient will be defined as a confirmed PR. Similarly, if a patient has two consecutive visit responses of CR, as long as there is no PD between CR visit then the patient will be defined as a confirmed CR.

a “Confirmed” response, for example a Confirmed CR means that the initial response (decrease in tumor size) as reported was seen again on a second later imaging scan.

TABLE 5
Monotherapy - PIK3CA Mutations

| | DKN-01 Monotherapy | | | |
|---|---|---|---|---|
| | In Recurrent | | In Recurrent | In Recurrent |
| | EEC | | EOC | MMMT |
| | (N = 29) | | (N = 14) | (N = 9) |
| PIK3CA | 13 | | 0 | 4 |
| Best Overall Response, n (%) | | | | |
| Complete Response (CR) | 1 | (7.7) | 0 | 0 |
| Confirmed CR | 1 | (7.7) | 0 | 0 |
| Partial Response (PR) | 1 | (7.7) | 0 | 0 |
| Confirmed PR | 1 | (7.7) | 0 | 0 |
| Stable Disease (SD) | 4 | (30.8) | 0 | 0 |
| Durable SD (>120 days) | 4 | (30.8) | 0 | 0 |
| Durable SD (>180 days) | 2 | (15.4) | 0 | 0 |
| Durable SD (>365 days) | 0 | | 0 | 0 |
| Progressive Disease (PD) | 7 | (53.8) | 0 | 4 (100) |

| | | | |
|---|---|---|---|
| Not Evaluable (NE) | 0 | 0 | 0 |

TABLE 6
Monotherapy - Non-PIK3CA Mutations

| | DKN-01 Monotherapy | | | |
|---|---|---|---|---|
| | In Recurrent | | In Recurrent | In Recurrent |
| | EEC | | EOC | MMMT |
| | (N = 29) | | (N = 14) | (N = 9) |
| Non PIK3CA | 16 | | 14 | 5 |
| Best Overall Response, n (%) | | | | |
| Complete Response (CR) | 0 | | 0 | 0 |
| Confirmed CR | 0 | | 0 | 0 |
| Partial Response (PR) | 0 | | 0 | 0 |
| Confirmed PR | 0 | | 0 | 0 |
| Stable Disease (SD) | 5 | (31.3) | 6 (42.9) | 2 (40.0) |
| Durable SD (>120 days) | 4 | (25.0) | 0 | 2 (40.0) |
| Durable SD (>180 days) | 4 | (25.0) | 0 | 2 (40.0) |
| Durable SD (>365 days) | 1 | (6.3) | 0 | 0 |
| Progressive Disease (PD) | 8 | (50.0) | 7 (50.0) | 3 (60.0) |
| Not Evaluable (NE) | 3 | (18.8) | 1 | 0 |

TABLE 7
Combination Therapy - PIK3CA Mutations

| | DKN-01 + Paclitaxel | | |
|---|---|---|---|
| | In Recurrent | In Recurrent | In Recurrent |
| | EEC | EOC | MMMT |
| | (N = 23) | (N = 19) | (N = 14) |
| PIK3CA | 10 | 1 | 5 |
| Best Overall Response, n (%) | | | |
| Complete Response (CR) | 0 | 0 | 0 |
| Confirmed CR | 0 | 0 | 0 |
| Partial Response (PR) | 0 | 0 | 2 (40.0) |
| Confirmed PR | 0 | 0 | 2 (40.0) |
| Stable Disease (SD) | 6 (60.0) | 0 | 0 |
| Durable SD (>120 days) | 6 (60.0) | 0 | 0 |
| Durable SD (>180 days) | 3 (30.0) | 0 | 0 |
| Durable SD (>365 days) | 1 (10.0) | 0 | 0 |
| Progressive Disease (PD) | 1 (10.0) | 1 (100) | 2 (40.0) |
| Not Evaluable (NE) | 3 (30.0) | 0 | 1 (20.0) |

TABLE 8
Combination Therapy - Non-PIK3CA Mutations

| | DKN-01 + Paclitaxel | | | | |
|---|---|---|---|---|---|
| | In Recurrent | | In Recurrent | | In Recurrent |
| | EEC | | EOC | | MMMT |
| | (N = 23) | | (N = 19) | | (N = 14) |
| Non PIK3CA | 13 | | 18 | | 9 |
| Best Overall Response, n (%) | | | | | |
| Complete Response (CR) | 0 | | 0 | | 0 |
| Confirmed CR | 0 | | 0 | | 0 |
| Partial Response (PR) | 0 | | 0 | | 0 |
| Confirmed PR | 0 | | 0 | | 0 |
| Stable Disease (SD) | 5 | (38.5) | 13 | (72.2) | 4 (44.4) |
| Durable SD (>120 days) | 3 | (23.1) | 4 | (22.2) | 0 |
| Durable SD (>180 days) | 1 | (7.7) | 2 | (11.1) | 0 |
| Durable SD (>365 days) | 0 | | 0 | | 0 |
| Progressive Disease (PD) | 8 | (61.5) | 5 | (27.8) | 4 (44.4) |

| | | | |
|---|---|---|---|
| Not Evaluable (NE) | 0 | 0 | 1 (11.1) |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of treating a subject suffering from a cancer, comprising the steps of:
obtaining a sample of a cancer cell from the subject;
determining a sequence of a phosphatidylinositol 3-kinase catalytic subunit (PIK3CA) protein in the sample; and
administering a first amount of a DKK1 inhibitor to the subject determined to have the sequence of PIK3CA protein (SEQ ID NO:23) that includes an activating mutation,
wherein the cancer is an epithelial endometrial cancer or an epithelial ovarian cancer or an MMMT.

2. A method of treating a cancer in a subject in need thereof, the method comprising:
administering a first amount of a DKK1 inhibitor to the subject, wherein the subject is determined to have an activating mutation of a phosphatidylinositol 3-kinase catalytic subunit (PIK3CA) protein (SEQ ID NO:23), and wherein the cancer is an epithelial endometrial cancer or an epithelial ovarian cancer or an MMMT.

3-4. (canceled)

5. The method of claim 2, wherein the DKK1 inhibitor is a DKK1 antibody or antigen binding-fragment thereof.

6. The method of claim 2, wherein the DKK1 antibody, or antigen binding-fragment thereof, comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO:1, LCDR2 has the amino sequence of SEQ ID NO:2, LCDR3 has the amino sequence of SEQ ID NO:3, HCDR1 has the amino sequence of SEQ ID NO:4, HCDR2 has the amino sequence of SEQ ID NO:5, and an HCDR3 has the amino sequence of SEQ ID NO:6.

7. The method of claim 6, wherein the LCVR comprises the amino acid sequence of SEQ ID NO:7 and the HCVR comprises the amino acid sequence of SEQ ID NO:8.

8. The method of claim 6, wherein the LCVR and HCVR comprise amino acid sequences selected from the group consisting of: (i) a LCVR comprising the amino acid sequence of SEQ ID NO:9 and a HCVR comprising the amino acid sequence of SEQ ID NO:10; (ii) a LCVR comprising the amino acid sequence of SEQ ID NO:11 and a HCVR comprising the amino acid sequence of SEQ ID NO:12; (iii) a LCVR comprising the amino acid sequence of SEQ ID NO:13 and a HCVR comprising the amino acid sequence of SEQ ID NO:10; (iv) a LCVR comprising the amino acid sequence of SEQ ID NO:14 and a HCVR comprising the amino acid sequence of SEQ ID NO:10.

9. The method of claim 8, wherein the LCVR comprises the amino acid sequence of SEQ ID NO:11 and the HCVR comprises the amino acid sequence of SEQ ID NO:12.

10. The method of claim 9, wherein the DKK1 antibody comprises a heavy chain and a light chain amino acid sequence selected from the group consisting of a) a heavy chain comprising the amino acid sequence of SEQ ID NO:19 and light chain comprising the amino acid sequence of SEQ ID NO:16, b) a heavy chain comprising the amino acid sequence of SEQ ID NO:17 and a light chain comprising the amino acid sequence of SEQ ID NO:18, c) a heavy chain comprising the amino acid sequence of SEQ ID NO:19 and a light chain comprising the amino acid sequence of SEQ ID NO:20, and d) a heavy chain comprising the amino acid sequence of SEQ ID NO:19 and a light chain comprising the amino acid sequence of SEQ ID NO:21.

11. The method of claim 5, wherein the DKK1 antibody comprises a heavy chain comprising the amino acid sequence of SEQ ID NO:17 and a light chain comprising the amino acid sequence of SEQ ID NO:18.

12. The method of claim 2, wherein the subject is a human.

13. The method of claim 2, further comprising administering to the subject a second amount of a second therapeutic agent.

14. The method of claim 13, wherein the second therapeutic agent is a taxane.

15. The method of claim 14, wherein the second agent is a paclitaxel.

16. The method of claim 13, wherein the DKK1 inhibitor is the DKN01 antibody, and the second therapeutic agent is paclitaxel.

17. The method of claim 13, further comprising administering to the subject a third amount of a third therapeutic agent.

18. The method of claim 17, wherein the second therapeutic agent is gemcitabine and the third therapeutic agent is a cisplatin.

19. The method of claim 2, wherein the mutation is at least one of N345D, H1047R, and E545K.

20. The method of claim 2, wherein the mutation is M1043L and amplification is present.

21. The method of claim 2, wherein the mutation is any one of the mutations of amino acid residues listed in Table 1.

22. The method of claim 2, wherein the mutation is any one of the mutations of amino acid residues listed in Table 1.

23. The method of claim 2, wherein the subject has undergone at least one prior therapy for the cancer being treated.
